(19) United States
(12) Patent Application Publication
(10) Pub. No.: US 2025/0282847 A1
(43) Pub. Date: Sep. 11, 2025

(54) CODON-OPTIMIZED NUCLEIC ACID ENCODING THE FVIII-BDD

(71) Applicant: JOINT STOCK COMPANY "BIOCAD", Saint Petersburg (RU)

(72) Inventors: Tatiana Evgenievna SHUGAEVA, Moscow (RU); Elena Veniaminovna VLASOVA, g. Sortavala (RU); Anastasiia Vladimirovna FOMINA, g. Ulyanovsk (RU); Mariya Pavlovna PEREPELKINA, Saint Petersburg (RU); Anna Nikolaevna STRELKOVA, g. Yaransk (RU); Pavel Mikhailovich GERSHOVICH, Saint Petersburg (RU); Pavel Andreevich IAKOVLEV, Saint Petersburg (RU); Dmitry Valentinovich MOROZOV, Saint Petersburg (RU)

(21) Appl. No.: 18/859,760

(22) PCT Filed: Apr. 18, 2023
(86) PCT No.: PCT/RU2023/050094

§ 371 Date: Oct. 24, 2024

(30) Foreign Application Priority Data
Apr. 28, 2022 (RU) 2022111734

(51) Int. Cl. C07K 14/755; A61K 38/37; A61K 48/00; A61P 7/04; C12N 15/86

(52) U.S. Cl. CPC C07K 14/755; A61K 38/37; A61K 48/005; A61P 7/04; C12N 15/86; C12N 2750/14143; C12N 2830/50

(57) ABSTRACT

The present application relates to the fields of genetics, gene therapy, and molecular biology. More specifically, the present invention relates to a codon-optimized nucleic acid that encodes the FVIII-BDD (B-domain deleted coagulation factor VIII) protein, to an expression cassette and a vector based thereon, to a host cell for producing FVIII-BDD, as well as to various uses of the above vector.

### FIELD OF THE INVENTION

The present application relates to the fields of genetics, gene therapy, and molecular biology. More specifically, the present invention relates to a codon-optimized nucleic acid that encodes the FVIII-BDD (B-domain deleted coagulation factor VIII) protein, to an expression cassette and a vector based thereon, to a host cell for producing FVIII-BDD, as well as to various uses of the above vector.

### BACKGROUND OF THE INVENTION

Haemophilia is a recessive, X-linked, inherited disorder causing a deficiency of one of the proteins involved in secondary hemostasis. Haemophilia A, or classic haemophilia, is the most common variant of haemophilia; it occurs in 1 out of 5000 newborn males (Report on the annual global survey 2019, WFH https://www.wfh.org/en/our-work-research-data/annual-global-survey) and is caused by a deficiency of coagulation factor VIII protein. According to the Russian Haemophilia Society, there are more than 6.5 thousand patients with haemophilia A in Russia (Report on the annual global survey 2019, WFH).

Coagulation factor VIII (FVIII) is a 280 kDa protein that is secreted into the blood mainly from sinusoidal liver epithelial cells (Fahs SA, Hille MT, Shi Q, Weiler H, Montgomery RR. A conditional knockout mouse model reveals endothelial cells as the principal and possibly exclusive source of plasma factor VIII/Blood. 2014 Jun. 12;123 (24): 3706-13. doi: 10.1182/blood-2014-02-555151. Epub 2014 Apr. 4. PMID: 24705491 and Everett LA, Cleuren AC, Khoriaty RN, Ginsburg D. Murine coagulation factor VIII is synthesized in endothelial cells/Blood. 2014 Jun. 12;123 (24): 3697-705. doi: 10.1182/blood-2014-02-554501. Epub 2014 Apr. 9. PMID: 24719406). Activated FVIII circulates in an organism as a heterodimer consisting of a heavy chain (A1, A2, B domains) and a light chain (A3, C1, C3 domains) bound to one another through non-covalent metal-dependent interactions. As a result of FVIII processing, only A1-A3, C1, C2 domains are present in the activated form of the protein. This fact contributed to the generation of a B-domain deleted recombinant FVIII (FVIII-BDD), which is not inferior in activity thereof to full-length FVIII (Pittman DD, Alderman EM, Tomkinson KN, Wang JH, Giles AR, Kaufman RJ. Biochemical, immunological, and in vivo functional characterization of B-domain-deleted factor VIII/Blood. 1993 Jun. 1;81 (11): 2925-35. PMID: 8499631). Unlike other proteins of the blood coagulation cascade, which mainly belong to proteases, FVIII is a glycoprotein. However, it plays a crucial role in the formation of a tenase complex that is necessary for the generation of activated coagulation factor X (FXa), the first member of the final common coagulation pathway, which ultimately leads to cross-linked fibrin formation.

Inversions of intron 1 or intron 22 of the FVIII gene originate more than half of cases of severe haemophilia A (Habart D, Kalabova D, Novotny M, Vorlova Z. Thirty-four novel mutations detected in factor VIII gene by multiplex CSGE: modeling of 13 novel amino acid substitutions/J Thromb Haemost. 2003 April;1 (4): 773-81. doi: 10.1046/j.1538-7836.2003.00149.x. PMID: 1287141). In other cases, abnormalities in the FVIII sequence are associated with various mutations, including antisense mutations, reading frame shifts, splice-site mutations, deletions and insertions.

The tendency to bleeding in haemophilia A is correlated with the defined FVIII activity and is classified as mild (0.05-0.40 IU/ml), moderate (0.01-0.05 IU/ml) or severe (<0.01 IU/ml). Patients with mild haemophilia typically experience abnormal bleeding only in connection with medical intervention or injuries. On the contrary, patients with moderate haemophilia exhibit prolonged bleeding reactions to relatively minor injuries, and patients with severe disease often have spontaneous bleeding. Severe haemophilia A manifests with spontaneous haemarthrosis, soft-tissue hematomas, retroperitoneal hemorrhage, intracerebral hemorrhage and delayed postoperative bleeding. Over time, complications from recurrent haemarthrosis and soft-tissue haematomas can result in severe arthropathy, joint contractures, and pseudotumours, leading to chronic diseases. The proportion of patients with mild, moderate and severe variants of haemophilia A is not precisely known, but recent epidemiological studies reported that approximately 60% of patients with haemophilia A have a severe variant (Report on the annual global survey 2019, WFH).

To date, lifelong replacement therapy in the form of injections of recombinant FVIII is the standard of care for patients with haemophilia A (Report on the annual global survey 2019, WFH). Despite the success achieved in the therapy of haemophilia, this approach has serious problems. Prophylactic replacement therapy in haemophilia A involves intravenous injections of recombinant FVIII every 3 days throughout the life of a patient with a severe variant of the disease. Such treatment approach is very expensive and does not guarantee the absence of complications primarily associated with haemarthrosis. In some cases, patients develop inhibitory antibodies. Inhibitory variants of haemophilia A are more typically observed in patients with severe disease and require the use of alternative approaches to the treatment and prophylaxis of the disease (Eckhardt CL, van der Bom JG, van der Naald M, Peters M, Kamphuisen PW, Fijnvandraat K. Surgery and inhibitor development in haemophilia A: a systematic review/J Thromb Haemost. 2011 October; 9 (10): 1948-58. doi: 10.1111/j.1538-7836.2011.04467.x. PMID: 21838755).

Gene therapy of haemophilia A by means of adeno-associated virus (AAV)-based viral (expression) vectors encoding the FVIII gene has shown excellent results in a series of preclinical and clinical studies (Bunting S, Zhang L, Xie L, Bullens S, Mahimkar R, Fong S, Sandza K, Harmon D, Yates B, Handyside B, Sihn CR, Galicia N, Tsuruda L, O'Neill CA, Bagri A, Colosi P, Long S, Vehar G, Carter B. Gene Therapy with BMN 270 Results in Therapeutic Levels of FVIII in Mice and Primates and Normalization of Bleeding in Hemophilic Mice/Mol Ther. 2018 Feb. 7;26 (2): 496-509. doi: 10.1016/j.ymthe.2017.12.009. Epub 2017 Dec. 14.PMID: 29292164 and Peyvandi F, Garagiola I. Clinical advances in gene therapy updates on clinical trials of gene therapy in haemophilia/Haemophilia. 2019 September;25 (5): 738-746. doi: 10.1111/hae.13816. Epub 2019 Jul. 8.PMID: 31282050). Unlike traditional approaches to the treatment of haemophilia, gene therapy using AAV allows maintaining the expression levels of extrinsic FVIII at a sufficient level for several years following a single administration of the therapeutic to patients (Long BR, Veron P, Kuranda K, Hardet R, Mitchell N, Hayes GM, Wong WY, Lau K, Li M, Hock MB, Zoog SJ, Vettermann C, Mingozzi F, Schweighardt B. Early Phase Clinical Immunogenicity of Valoctocogene Roxaparvovec, an AAV5-Mediated Gene Therapy for Haemophilia A/Mol Ther. 2021 Feb. 3;29 (2): 597-610. doi: 10.1016/j.ymthe.2020.12.008. Epub 2020 Dec. 10. PMID: 33309883).

To date, no gene therapy product has been registered in the world for the treatment of haemophilia A.

Thus, there is a need to develop a gene therapy product for the treatment of haemophilia A, as well as solutions that will improve the efficacy of the gene therapy product for the treatment of haemophilia A.

### Disclosure of the Essence of the Invention

One of the urgent purposes of research in the area of development of effective gene therapy is codon optimization of genes of interest in vectors to achieve the maximum level of production of the protein of interest, which, in turn, will allow using lower doses of the vector to achieve a significant effect.

The authors of the invention surprisingly found that a codon-optimized nucleic acid that encodes the FVIII-BDD (B-domain deleted coagulation factor VIII) protein with the amino acid sequence of SEQ ID NO: 11 and includes a nucleotide sequence that is selected from the group: SEQ ID NO: 2 (variant FVIII-BDDco-v1), SEQ ID NO: 3 (variant FVIII-BDDco-v2), SEQ ID NO: 4 (variant FVIII-BDDco-v3) or SEQ ID NO: 5 (variant FVIII-BDDco-v4) surprisingly causes increased levels of FVIII-BDD gene expression, increased levels of coagulation factor VIII-BDD protein production, as well as to increased levels of coagulation factor VIII-BDD protein activity with fold increase as compared to a wild-type gene encoding the coagulation factor VIII-BDD (FVIII-BDD-wt). These variants of the codon-optimized nucleic acid according to the invention with a nucleotide sequence selected from the group: SEQ ID NO: 2 (variant FVIII-BDDco-v1), SEQ ID NO: 3 (variant FVIII-BDDco-v2), SEQ ID NO: 4 (variant FVIII-BDDco-v3) or SEQ ID NO: 5 (variant FVIII-BDDco-v4) are included in the expression cassette and the vector based thereon.

### Definitions and General Methods

Unless defined otherwise herein, all technical and scientific terms used in connection with the present invention will have the same meaning as is commonly understood by those skilled in the art.

Furthermore, unless otherwise required by context, singular terms shall include plural terms, and the plural terms shall include the singular terms. Typically, the present classification and methods of cell culture, molecular biology, immunology, microbiology, genetics, analytical chemistry, organic synthesis chemistry, medical and pharmaceutical chemistry, as well as hybridization and chemistry of protein and nucleic acids described herein are well known by those skilled and widely used in the art. Enzyme reactions and purification methods are performed according to the manufacturer's guidelines, as is common in the art, or as described herein.

The terms “naturally occurring”, “native”, or “wild-type” are used to describe an object that can be found in nature as distinct from being artificially produced. For example, a protein or nucleotide sequence present in an organism, including in a virus, which can be isolated from a source in nature and that has not been intentionally modified by a person in the laboratory, is naturally occurring.

As used in the present description and claims that follow, unless otherwise dictated by the context, the words “include” and “comprise”, or variations thereof such as “includes”, “including”, “comprises”, or “comprising”, will be understood to imply the inclusion of a stated integer or group of integers but not the exclusion of any other integer or group of integers.

### Protein (Peptide)

As used in the present description, the terms “peptide”, “polypeptide” and “protein” are used interchangeably, and they refer to a compound consisting of amino acid residues that are covalently linked by peptide bonds. The polypeptides include natural peptides, recombinant peptides, synthetic peptides, or a combination thereof.

### Nucleic Acid Molecules

The terms “nucleic acid”, “nucleic sequence”, “nucleic acid sequence”, “polynucleotide”, “oligonucleotide”, “polynucleotide sequence” and “nucleotide sequence”, used interchangeably in the present description, mean a precise sequence of nucleotides, modified or not, determining a fragment or a region of a nucleic acid, containing unnatural nucleotides or not, and being either a double-strand DNA or RNA, a single-strand DNA or RNA, or transcription products of said DNAs.

As used in the present description, polynucleotides include, as non-limiting examples, all nucleic acid sequences which are obtained by any means available in the art, including, as non-limiting examples, recombinant means, i.e. the cloning of nucleic acid sequences from a recombinant library or a cell genome, using ordinary cloning technology and PCR and the like, and by synthetic means.

It should also be included here that the present invention does not relate to nucleotide sequences in their natural chromosomal environment, i.e. in a natural state. The sequences of the present invention have been isolated and/or purified, i.e., they were sampled directly or indirectly, for example by copying, their environment having been at least partially modified. Thus, isolated nucleic acids obtained by recombinant genetics, by means, for example, of host cells, or obtained by chemical synthesis should also be mentioned here.

Unless otherwise indicated, the term nucleotide sequence encompasses its complement. Thus, a nucleic acid having a particular sequence should be understood as one which encompasses the complementary strand thereof with the complementary sequence thereof.

### Vector

The term “vector” as used herein means a nucleic acid molecule capable of transporting another nucleic acid to which it has been linked. Furthermore, the term “vector” herein refers to a recombinant viral particle capable of transporting a nucleic acid.

As used in the present description, the term “expression” is defined as the transcription and/or translation of a particular nucleotide sequence driven by its promoter.

### Use

“Gene therapy” is the insertion of genes into subject's cells and/or tissues to treat a disease, typically hereditary diseases, in which a defective mutant allele is replaced with a functional one.

“Treat”, “treatment” and “therapy” refer to a method of alleviating or abrogating a biological disorder and/or at least one of attendant symptoms thereof.

The terms “subject”, “patient”, “individual”, and the like are used interchangeably in the present description, and they refer to any animal which is amenable to the methods described in the present description. In certain non-limiting embodiments, the subject, patient or individual is a human. Said subject may be either male or female, of any age.

“Therapeutically effective amount” or “effective amount” refers to that amount of the therapeutic agent being administered which will relieve to some extent one or more of the symptoms of the disease being treated.

### DETAILED DESCRIPTION OF THE INVENTION

### Nucleic Acid

In one aspect, the present invention relates to an isolated codon-optimized nucleic acid that encodes the FVIII-BDD (B-domain deleted coagulation factor VIII) protein with the amino acid sequence of SEQ ID NO: 11 and includes a nucleotide sequence that is selected from the group: SEQ ID NO: 2, SEQ ID NO: 3, SEQ ID NO: 4 or SEQ ID NO: 5.

An “isolated” nucleic acid molecule is one which is identified and separated from at least one nucleic acid molecule-impurity. An isolated nucleic acid molecule is different from the form or set in which it is found under natural conditions. Thus, an isolated nucleic acid molecule is different from a nucleic acid molecule that exists in cells under natural conditions.

One of the properties of the genetic code is degeneracy, i.e. the ability of different codons (trinucleotides) to encode the same amino acid. Such codons that are translated to the same amino acid are called synonymous codons. In natural sequences, one of the synonymous codons is selected randomly in the course of evolution, but the frequencies of usage of synonymous codons are different: each amino acid has more and less preferred ones. Codon optimization is a widely used technique to amplify the production of protein molecules, which provides a rational mapping of one of suitable synonymous codons to each amino acid in a protein sequence. One of the common principles of codon optimization involves the usage of the most frequent codons, whereas other approaches were introduced later, such as harmonization (reproduction of distribution of codon usage frequencies), but they do not always increase productivity. In addition to codon frequencies, the sequence GC content (ratio of guanine and cytosine to the total length of the sequence) may affect the production efficiency, in particular, it was shown that high GC content is associated with increased mRNA levels in mammalian cells (Grzegorz Kudla ET AL., High Guanine and Cytosine Content Increases mRNA Levels in Mammalian Cells, June 2006, Volume 4, Issue 6, e180, pp. 933-942). It is further worth noting that stable secondary structure elements of mRNA, i.e. those having a low free folding energy, may reduce the efficiency.

Different variants of codon-optimization of the sequence of a gene of interest may lead to the following (as compared to a wild-type gene):
a) expression levels of the genes of interest will be slightly increased;
b) expression levels of the genes of interest will be significantly increased;
c) expression levels of the genes of interest will remain approximately at the same level;
d) expression levels of the genes of interest will be lowered.

The above codon-optimized nucleic acid according to the invention was produced by codon-optimizing a wild-type nucleic acid with the nucleotide sequence of SEQ ID NO: 1.

As a result of codon optimization of the FIX-BDD protein-encoding wild-type nucleic acid having the nucleotide sequence of SEQ ID NO: 1, a number of codon-optimized nucleic acids were produced, which were further tested for the level of protein production as compared to the control FVIII-BDD-wt (wild-type nucleic acid with SEQ ID NO:1).

All codon-optimized nucleic acids caused an increased level of FVIII-BDD protein production as compared to the wild-type nucleic acid; further, a number of codon-optimized nucleic acids caused a slight increase in the level of FVIII-BDD protein production as compared to the wild-type nucleic acid. In turn, the codon-optimized nucleic acid according to the invention that is selected from the group: SEQ ID NO: 2 (variant FVIII-BDDco-v1), SEQ ID NO: 3 (variant FVIII-BDDco-v2), SEQ ID NO: 4 (variant FVIII-BDDco-v3) or SEQ ID NO: 5 (variant FVIII-BDDco-v4) surprisingly causes increased levels of FVIII-BDD gene expression, increased levels of coagulation factor VIII-BDD protein production, as well as to increased levels of coagulation factor VIII-BDD protein activity with fold increase as compared to a wild-type gene (FVIII-BDD-wt).

Expression Cassette. Expression Vector.

In one aspect, the present invention relates to an expression cassette comprising any one of the above codon-optimized nucleic acids encoding the FVIII-BDD protein.

The term “cassette which expresses” or “expression cassette”, as used herein, refers in particular to a DNA fragment that is capable, in an appropriate setting, of triggering the expression of a polynucleotide encoding a polypeptide of interest, the sequence of which is included in said expression cassette. When introduced into a host cell, the expression cassette is, inter alia, capable of engaging cellular mechanisms to transcribe the polynucleotide encoding the polypeptide of interest into RNA that is then typically further processed and eventually translated into the polypeptide of interest. The expression cassette may be contained in an expression vector.

The expression cassette of the present invention comprises a promoter as an element. The term “promoter” as used herein refers in particular to a DNA element that promotes the transcription of a polynucleotide to which the promoter is operably linked. The promoter may further form part of a promoter/enhancer element. Although the physical boundaries between the “promoter” and “enhancer” elements are not always clear, the term “promoter” typically refers to a site on the nucleic acid molecule to which an RNA polymerase and/or any associated factors binds and at which transcription is initiated. Enhancers potentiate promoter activity temporally as well as spatially. Many promoters are known in the art to be transcriptionally active in a wide range of cell types. Promoters can be divided into two classes, those that function constitutively and those that are regulated by induction or derepression. The both classes are suitable for protein expression. Promoters that are used for high-level production of polypeptides in eukaryotic cells and, in particular, in mammalian cells, should be strong and preferably active in a wide range of cell types. Strong constitutive promoters which are capable of driving expression in many cell types are well known in the art and, therefore, it is not herein necessary to describe them in detail.

According to one embodiment of the invention, the HLP promoter is used in the expression cassette of the present invention.

In some embodiments, the expression cassette includes the following elements in the 5′-end to 3′-end direction:
a left-hand (first) ITR (inverted terminal repeats);
a promoter;
a nucleic acid encoding a signal peptide;
any one of the above codon-optimized nucleic acids encoding the FVIII-BDD protein;
a polyadenylation signal;
a right-hand (second) ITR.

The above structural elements of the expression cassette are operably linked to one another.

As used herein, the term “operably linked” refers to a linkage of polynucleotide (or polypeptide) elements in a functional relationship. A nucleic acid is “operably linked” when it is present in functional relationship conditions with another nucleic acid sequence. For example, a transcription regulatory sequence is operably linked to a coding sequence if it affects the transcription of said coding sequence. The term “operably linked” means that the DNA sequences being linked are typically contiguous and, where it is necessary to join two protein coding regions, are also contiguous and are present in the reading frame.

In some embodiments, the expression cassette includes a left-hand (first) ITR with the nucleotide sequence of SEQ ID NO: 13.

In some embodiments, the expression cassette includes an HLP promoter with the nucleotide sequence of SEQ ID NO: 14.

In some embodiments, the expression cassette includes a polyadenylation signal with the nucleotide sequence of SEQ ID NO: 15.

In some embodiments, the expression cassette includes a right-hand (second) ITR with the nucleotide sequence of SEQ ID NO: 16.

In some embodiments, the nucleic acid encoding a signal peptide has a nucleotide sequence that is selected from the group: SEQ ID NO: 17, SEQ ID NO: 18, SEQ ID NO: 19, SEQ ID NO: 20 or SEQ ID NO: 21.

In some embodiments, the codon-optimized nucleic acid encoding the FVIII-BDD protein with a signal peptide (FVIII-BDDco-v1 variant) has the nucleotide sequence of SEQ ID NO: 7.

In some embodiments, the codon-optimized nucleic acid encoding the FVIII-BDD protein with a signal peptide (FVIII-BDDco-v2 variant) has the nucleotide sequence of SEQ ID NO: 8.

In some embodiments, the codon-optimized nucleic acid encoding the FVIII-BDD protein with a signal peptide (FVIII-BDDco-v3 variant) has the nucleotide sequence of SEQ ID NO: 9.

In some embodiments, the codon-optimized nucleic acid encoding the FVIII-BDD protein with a signal peptide (FVIII-BDDco-v4 variant) has the nucleotide sequence of SEQ ID NO: 10.

In some embodiments, the expression cassette includes the following elements in the 5′-end to 3′-end direction:
a left-hand (first) ITR (inverted terminal repeats) with the nucleotide sequence of SEQ ID NO: 13;
a promoter with the nucleotide sequence of SEQ ID NO: 14;
a nucleic acid encoding a signal peptide that has a nucleotide sequence that is selected from the group: SEQ ID NO: 17, SEQ ID NO: 18, SEQ ID NO: 19, SEQ ID NO: 20 or SEQ ID NO: 21;
any one of the above codon-optimized nucleic acids encoding the FVIII-BDD protein;
a polyadenylation signal with the nucleotide sequence of SEQ ID NO: 15;
a right-hand (second) ITR with the nucleotide sequence of SEQ ID NO: 16.

In some embodiments, the expression cassette includes the following elements in the 5′-end to 3′-end direction:
a left-hand (first) ITR (inverted terminal repeats) with the nucleotide sequence of SEQ ID NO: 13;
a promoter with the nucleotide sequence of SEQ ID NO: 14;
a codon-optimized nucleic acid encoding the FVIII-BDD protein with a signal peptide, the codon-optimized nucleic acid having a nucleotide sequence that is selected from the group: SEQ ID NO: 7, SEQ ID NO: 8, SEQ ID NO: 9 or SEQ ID NO: 10;
a polyadenylation signal with the nucleotide sequence of SEQ ID NO: 15;
a right-hand (second) ITR with the nucleotide sequence of SEQ ID NO: 16.

In one aspect, the present invention relates to an expression vector that includes any one of the above codon-optimized nucleic acids encoding the FVIII-BDD protein, or any one of the above expression cassettes.

In some embodiments of the invention, the vector is a plasmid, i.e. a circular double stranded piece of DNA into which additional DNA segments may be inserted.

In some embodiments of the invention, the vector is a viral (expression) vector, wherein additional DNA segments may be inserted into the viral genome.

In some embodiments of the invention, vectors are capable of autonomous replication in a host cell into which they are introduced (e.g. bacterial vectors having a bacterial site of replication origin and episomal vectors). In further embodiments of the invention, vectors (e.g. non-episomal vectors) may be integrated into the genome of a host cell upon introduction into a host cell, and thereby are replicated along with the host gene. Moreover, certain vectors are capable of directing the expression of genes to which they are operably linked. Such vectors are referred to herein as “recombinant expression vectors” (or simply, “expression vectors”).

Expression vectors include plasmids, retroviruses, adenoviruses, adeno-associated viruses (AAVs), plant viruses, such as cauliflower mosaic virus, tobacco mosaic virus, cosmids, YACs, EBV, and the like. DNA molecules may be inserted into a vector such that transcriptional and translational control sequences within the vector serve their intended function of regulating the transcription and translation of the DNA. An expression vector and expression control sequences may be chosen to be compatible with the expression host cell used. DNA molecules may be introduced into the expression vector by standard methods (e.g. ligation of complementary restriction sites, or blunt end ligation if no restriction sites are present).

In some embodiments, the expression vector is a recombinant adeno-associated virus (AAV).

In some embodiments, the AAV is selected from a group including the following AAV serotypes: AAV1, AAV2, AAV3, AAV4, AAV5, AAV6, AAV7, AAV8, AAV9, AAV10 AAV11, AAV12, AAV13, AAV14, AAV15, AAV16, rAAV.rh8, rAAV.rhlO, rAAV.rh20, rAAV.rh39, rAAV.Rh74, rAAV.RHM4-1, AAV.hu37, rAAV.Anc80, rAAV.Anc80L65, rAAV.7m8, rAAV.PHP.B, rAAV2.5, rAAV2tYF, rAAV3B, rAAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15 or AAV.HSC16.

In some embodiments of the invention, the vector or cassette may include an expression control sequence. The term “expression control sequence” as used in the present description refers to polynucleotide sequences that are necessary to effect the expression and processing of coding sequences to which they are inserted. It will be understood by those skilled in the art that the design of an expression vector or cassette, including the selection of expression control sequences, may depend on such factors as the choice of the type of a host cell to be transformed, the required level of expression of a protein, and so forth. The expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include a promoter, a ribosome binding site, and transcription termination sequences; in eukaryotes, such control sequences typically include promoters and transcription termination sequences. Preferred expression control sequences for an expression host cell in a mammal include viral elements that ensure high levels of protein expression in mammalian cells, such as promoters and/or enhancers derived from a retroviral LTR, cytomegalovirus (CMV) (such as a CMV promoter/enhancer), simian virus 40 (SV40) (such as a SV40 promoter/enhancer), adenovirus, (e.g. the major late promoter adenovirus (AdMLP)), polyomavirus and strong mammalian promoters such as TTR promoter, native immunoglobulin promoter, actin promoter, and HLP (hybrid liver-specific promoter). Expression control sequences encompass at least all components whose presence is important for expression and processing.

In addition to the above genes and expression control sequences, recombinant expression vectors of the invention may carry additional sequences, such as sequences that regulate replication of a vector in host cells (e.g. origins of replication) and selectable marker genes. The selectable marker gene facilitates the selection of host cells into which a vector or cassette has been introduced.

In one embodiment of the present invention, the expression vector relates to a vector comprising one or more polynucleotide sequences of interest, genes of interest, or transgenes that are flanked by parvoviral sequences or inverted terminal repeat (ITR) sequences.

Neither the cassette nor the vector of the invention comprises nucleotide sequences of genes encoding non-structural proteins (Rep) and structural proteins (Cap) of the adeno-associated virus.

### Host Cell

In one aspect, the present invention relates to a host cell for producing the FVIII-BDD protein or for producing any one of the above expression vectors, which comprises any one of the above codon-optimized nucleic acids encoding the FVIII-BDD protein.

The term “host cell” as used herein refers to a cell into which a recombinant expression vector or cassette according to the invention has been introduced. The present invention relates to host cells, which may include, for example, the above-described vector according to the invention. It should be understood that “host cell” refers not only to a particular subject cell but to the progeny of such cell as well. Since modifications may occur in succeeding generations due to either mutation or environmental influences, such progeny may not, in fact, be identical to a parental cell; however, such cells are still included within the scope of the term “host cell” as used herein.

Expression vectors or cassettes according to the invention may be used for transfection of a mammalian cell, plant cell, bacterial or yeast host cell. Transfection may be carried out by any known technique of introducing polynucleotides into a host cell. Methods for introduction of heterologous polynucleotides into mammalian cells are well known in the art and include dextran-mediated transfection, cationic polymer-nucleic acid complex transfection, calcium phosphate precipitation, polybrene-mediated transfection, protoplast fusion, encapsulation of the polynucleotides in liposomes, and direct microinjection of DNA into nuclei. In addition, the nucleic acid molecules may be introduced into mammalian cells by viral (expression) vectors.

Mammalian cell lines used as hosts for transformation are well known in the art and include a plurality of immortalized cell lines available. These include, e.g., Chinese hamster ovary (CHO) cells, NSO cells, SP2 cells, HEK-293T cells, FreeStyle 293 cells (Invitrogen), NIH-3T3 cells, HeLa cells, baby hamster kidney (BHK) cells, African green monkey kidney cells (COS), human hepatocellular carcinoma cells (e.g., Hep G2), A549 cells, SK-HEP1, HUH7, Hep-RG and a number of other cell lines. Cell lines are selected by determining which cell lines have high expression levels and provide for necessary characteristics of the protein being produced. Other cell lines that may be used are insect cell lines, such as Sf9 or Sf21 cells. When the recombinant expression vectors of the invention are introduced into mammalian host cells, the FVIII-BDD protein is produced by culturing the host cells for a period of time sufficient to express the fusion protein in host cells, or, more preferably, secrete the fusion protein into the culture medium in which the host cells are cultured. The fusion protein may be isolated from culture medium using standard protein purification techniques. Plant host cells include e.g. Nicotiana, Arabidopsis, duckweed, corn, wheat, potato, etc. Bacterial host cells include Escherichia and Streptomyces species. Yeast host cells include Schizosaccharomyces pombe, Saccharomyces cerevisiae and Pichia pastoris.

The above host cell does not refer to a host cell produced using human embryos.

The above host cell does not refer to a host cell produced by modifying the genetic integrity of human germline cells.

### Pharmaceutical Composition

In one aspect, the present invention relates to a pharmaceutical composition for delivering the FVIII-BDD gene to target cells, which includes any one of the above expression vectors or cassettes.

In some embodiments, the pharmaceutical composition for delivering the FVIII-BDD gene to target cells includes any one of the above expression vectors or cassettes in combination with one or more pharmaceutically acceptable excipients.

The active substance in the above composition is present in an effective amount, for example, in a biologically effective amount.

“Pharmaceutical composition” means a composition comprising any one of the above expression vectors according to the invention and at least one of components selected from the group consisting of pharmaceutically acceptable and pharmacologically compatible excipients, fillers, solvents, diluents, carriers, auxiliary agents, distributing agents, or delivery agents.

The pharmaceutical compositions of the present invention and methods of preparation thereof will be undoubtedly apparent to those skilled in the art. The pharmaceutical compositions should preferably be manufactured in compliance with the GMP (Good Manufacturing Practice) requirements.

In some embodiments of the pharmaceutical composition, it may include a buffer composition, tonicity agents (osmolyte or osmotic agent), stabilizers and/or solubilizers.

“Pharmaceutically acceptable” means a material that does not have biological or other negative side effects, for example, the material can be administered to a subject without causing any undesirable biological effects. Thus, such pharmaceutical compositions may be used, for example, in ex vivo transfection of a cell or in in vivo administration of any one of the above expression vectors of the invention directly to a subject.

The term “excipient” is used herein to describe any ingredient other than the above ingredients of the invention. These are substances of inorganic or organic nature which are used in the pharmaceutical production/manufacturing in order to give drug products the necessary physicochemical properties.

The pharmaceutical composition according to the invention is a stable composition.

The pharmaceutical composition is “stable” if the active agent retains physical stability and/or chemical stability and/or biological activity thereof during the specified shelf life at storage temperature, for example, of 2-8° C. Preferably, the active agent retains both physical and chemical stability, as well as biological activity. Storage period is adjusted based on the results of stability test in accelerated or natural aging conditions.

In some embodiments, the pharmaceutical composition is a solution for intravenous administration.

In some embodiments, the pharmaceutical composition is a concentrate for the preparation of a solution for intravenous administration.

### Use

In one aspect, the present invention relates to the use of any one of the above expression cassettes or vectors or the above composition to deliver the FVIII-BDD gene to target cells.

In one aspect, the present invention relates to the use of any one of the above expression cassettes or vectors or the above composition to provide the FVIII-BDD protein to a subject who has haemophilia A and/or does not have functional copies of the FVIII gene.

The lack of functional copies of the FVIII gene refers to inactivating mutations or deletions in all copies of the FVIII gene in the genome, which result in the loss or defect of the function of the FVIII gene.

In one aspect, the present invention relates to a method for providing the FVIII-BDD protein to a subject with haemophilia A, comprising introducing a therapeutically effective amount of any one of the above expression vectors or the above composition into the cells of the subject in need thereof.

In one aspect, the present invention relates to a method of delivering the FVIII-BDD gene to target cells of a subject with haemophilia A, comprising introducing any one of the above expression vectors or the above composition into the cells of the subject.

A subject in need of delivering the FVIII-BDD gene to target cells, or a subject in need of being provided with the FVIII-BDD protein refers to a subject who has haemophilia A, or to a subject who has the deficiency of coagulation factor FVIII, or to a subject who has inactivating mutations or deletions in the FVIII gene that lead to loss of or defect in the function of the FVIII gene.

In one aspect, the present invention relates to the use of any one of the above expression vectors or the above composition for treating haemophilia A in a subject that has haemophilia A.

In one aspect, the present invention relates to a method for treating haemophilia A in a subject, comprising administering a therapeutically effective amount of any one of the above expression vectors or the above composition into a subject that has haemophilia A.

In some embodiments, haemophilia A is severe haemophilia A (<1% factor VIII activity) or moderate haemophilia A (1-5% factor VIII activity).

Exemplary modes of administration include topical application, intranasal, inhalation, transmucosal, transdermal, enteral (e.g. oral, rectal), parenteral (e.g. intravenous, subcutaneous, intradermal, intramuscular) administrations, as well as direct tissue or organ injections.

In some embodiments of the use, any one of the above expression vectors or the above composition is administered to the subject as an intravenous infusion.

Any one of the above expression vectors are administered into an organism in an effective amount. Any one of the above expression vectors is preferably administered into an organism in a biologically effective amount. A “biologically effective” amount of the expression vector is an amount that is sufficient to cause cell transduction and expression of a nucleic acid sequence in the cell. If the expression vector is administered into a cell in vivo, a “biologically-effective” amount of the expression vector is an amount that is sufficient to cause the transduction of target cells and expression of the nucleic acid sequence in a target cell.

Dosages of any one of the above expression vectors according to the present invention will depend on the mode of administration of a particular vector, and they can be determined in a routine manner.

The cell for administering any one of the above expression cassettes or vectors according to the invention may be a cell of any type, including but not limited to epithelial cells (e.g. skin, respiratory and gut epithelial cells), hepatic cells, muscle cells, pancreatic cells (including islet cells), hepatic cells, spleen cells, fibroblasts, endothelial cells, and the like.

Any one of the above expression cassettes or vectors according to the invention is not used to modify the genetic integrity of human germ line cells.

In some embodiments of the use, any one of the above expression vectors according to the present invention, as well as a product based thereon, are used as monotherapy.

In some embodiments of the use, any one of the above expression vectors according to the present invention, as well as a product based thereon, are used in combination with replacement therapy with coagulation factor concentrates, desmopressin and/or fibrinolysis inhibitors.

In some embodiments of the use, any one of the above expression vectors according to the present invention, as well as a product based thereon, are used in combination with a monoclonal antibody (for example, emicizumab).

In some embodiments of the use, any one of the above expression vectors according to the present invention, as well as a therapeutic based thereon, are used in combination with RNA interference therapeutics (for example, fitusiran).

In some embodiments of the use, any one of the above expression vectors according to the present invention, as well as a product based thereon, are administered into a subject once.

In some embodiments of the use, any one of the above expression vectors according to the present invention, as well as a product based thereon, are administered into a subject repeatedly.

### BRIEF DESCRIPTION OF DRAWINGS

FIG. 1 is a scheme of plasmid pAAV-hFVIII-BDD comprising the human coagulation factor VIII (FVIII) gene sequence selected from SEQ ID NO:1, SEQ ID NO:2, SEQ ID NO:3, SEQ ID NO:4 or SEQ ID NO:5. The plasmid sequence further comprises the following elements:
AmpR is a beta-lactamase gene that provides resistance to ampicillin;
pUC origin is a pUC replication origin in bacteria;
ITR is inverted terminal repeats;
HLP promoter is a synthetic tissue-specific promoter;
sPA is a synthetic polyadenylation signal sequence, for increasing mRNA stability.

FIG. 2 is a graph that shows increased level of FVIII-BDD protein production into culture fluid following transfection with codon-optimized nucleic acids encoding the FVIII-BDD (B-domain deleted coagulation factor VIII) protein as compared to a wild-type nucleic acid encoding the FVIII-BDD protein.

FVIII-BDD protein level following cell transfection:
1—with a wild-type nucleic acid that encodes the FVIII-BDD protein, with the nucleotide sequence of SEQ ID NO: 1.
2—with a codon-optimized nucleic acid that encodes the FVIII-BDD protein, with the nucleotide sequence of SEQ ID NO: 2.
3—with a codon-optimized nucleic acid that encodes the FVIII-BDD protein, with the nucleotide sequence of SEQ ID NO: 3.
4—with a codon-optimized nucleic acid that encodes the FVIII-BDD protein, with the nucleotide sequence of SEQ ID NO: 4.
5—with a codon-optimized nucleic acid that encodes the FVIII-BDD protein, with the nucleotide sequence of SEQ ID NO: 5.

FIG. 3 is a graph that shows increased level of FVIII-BDD protein activity in culture fluid following transfection with codon-optimized nucleic acids encoding the FVIII-BDD protein (B-domain deleted coagulation factor VIII) as compared to a wild-type nucleic acid encoding the FVIII-BDD protein.

FVIII-BDD activity level following cell transfection:
1—with a wild-type nucleic acid that encodes the FVIII-BDD protein, with the nucleotide sequence of SEQ ID NO: 1.
2—with a codon-optimized nucleic acid that encodes the FVIII-BDD protein, with the nucleotide sequence of SEQ ID NO: 2.
3—with a codon-optimized nucleic acid that encodes the FVIII-BDD protein, with the nucleotide sequence of SEQ ID NO: 3.
4—with a codon-optimized nucleic acid that encodes the FVIII-BDD protein, with the nucleotide sequence of SEQ ID NO: 4.
5—with a codon-optimized nucleic acid that encodes the FVIII-BDD protein, with the nucleotide sequence of SEQ ID NO: 5.

FIG. 4 is a graph that shows increased level of FVIII-BDD protein production in case of in vitro delivery of the nucleic acids in the form of an AAV expression vector comprising a codon-optimized nucleic acid encoding the FVIII-BDD protein (B-domain deleted coagulation factor VIII) as compared to an AAV expression vector comprising a wild-type nucleic acid encoding the FVIII-BDD protein.

FVIII-BDD protein level following cell transduction:
1—control solution free of AAV (negative control).
2—AAV serotype 6 expression vectors comprising codon-optimized nucleic acid that encodes the FVIII-BDD protein, with the nucleotide sequence of SEQ ID NO: 2.
3—AAV serotype 6 expression vectors comprising codon-optimized nucleic acid that encodes the FVIII-BDD protein, with the nucleotide sequence of SEQ ID NO: 5.
4—AAV serotype 5 expression vectors comprising codon-optimized nucleic acid that encodes the FVIII-BDD protein, with the nucleotide sequence of SEQ ID NO: 2.

FIG. 5 is a graph that shows increased level of FVIII-BDD protein activity in case of in vitro delivery of the nucleic acids in the form of an AAV expression vector comprising a codon-optimized nucleic acid encoding the FVIII-BDD protein (B-domain deleted coagulation factor VIII) as compared to an AAV expression vector comprising a wild-type nucleic acid encoding the FVIII-BDD protein.

FVIII-BDD activity level following cell transduction:
1—control solution free of AAV (negative control).
2—AAV serotype 6 expression vectors comprising codon-optimized nucleic acid that encodes the FVIII-BDD protein, with the nucleotide sequence of SEQ ID NO: 2.
3—AAV serotype 6 expression vectors comprising codon-optimized nucleic acid that encodes the FVIII-BDD protein, with the nucleotide sequence of SEQ ID NO: 5.
4—AAV serotype 5 expression vectors comprising codon-optimized nucleic acid that encodes the FVIII-BDD protein, with the nucleotide sequence of SEQ ID NO: 2.

FIG. 6 is a graph that shows increased level of the FVIII-BDD protein in case of in vivo delivery of a codon-optimized nucleic acid encoding the FVIII-BDD protein to B6.129S-F8tm1Smoc (HemA) mice in the form of an AAV expression vector.

FVIII-BDD protein levels in blood plasma of animals following injection:
1—control solution free of AAV (negative control).
2—AAV serotype 5 expression vector comprising codon-optimized nucleic acid that encodes the FVIII-BDD protein, with the nucleotide sequence of SEQ ID NO: 2.
3—AAV serotype 6 expression vectors comprising codon-optimized nucleic acid that encodes the FVIII-BDD protein, with the nucleotide sequence of SEQ ID NO: 2.

### EXAMPLES

The following examples are provided for better understanding of the invention. These examples are for purposes of illustration only and are not to be construed as limiting the scope of the invention in any manner.

All publications, patents, and patent applications cited in this specification are incorporated herein by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended embodiments.

### Materials and General Methods

### Recombinant DNA Techniques

Standard methods were used to manipulate DNA as described in Sambrook, J. et al, Molecular cloning: A laboratory manual; Cold Spring Harbor Laboratory Press, Cold Spring Harbor, New York, 1989. The molecular biological reagents were used according to the manufacturer protocols. Briefly, plasmid DNA was produced for further manipulation in E. coli cells grown under selective antibiotic pressure so that the plasmids were not lost in the cell population. We isolated the plasmid DNA from cells using commercial kits, measured the concentration, and used it for cloning by restriction endonuclease treatment or PCR amplification. The DNA fragments were ligated to each other using ligases and transformed into bacterial cells for the selection of clones and further production. All resulting genetic constructs were confirmed by restriction patterns and complete Sanger sequencing.

### Gene Synthesis

Desired gene segments were prepared from oligonucleotides made by chemical synthesis. Gene fragments of 300 to 1000 bp long, which were flanked by unique restriction sites, were collected by renaturing oligonucleotides on top of each other, followed by PCR amplification from border primers. As a result, a mixture of fragments was produced, including the desired one. The fragments were cloned at restriction sites into intermediate vectors, following which the DNA sequences of the subcloned fragments were confirmed by DNA sequencing.

### Dna Sequence Determination

DNA sequences were determined by Sanger sequencing. DNA and protein sequences were analyzed and sequence data was processed in SnapGene Viewer 4.2 or higher for sequence creation, mapping, analysis, annotation and illustration.

### Culturing Cell Cultures

The experiments used the following cell lines: HEK293 (Human Embryonic Kidney clone 293), SK-HEP1 (human adenocarcinoma endothelial cell line) and HUH7 (human hepatocellular carcinoma cell lines). The suspended HEK293 cells used to produce AAV were cultured under standard conditions at 37° C. and 5% CO₂ on a complete culture medium without FBS and antibiotic. The adherent SK-HEP1 cells used to test the efficacy of AAV products were cultured under standard conditions at 37° C. and 5% CO₂, on a complete EMEM medium supplemented with 10% FBS, antibiotic/antimycotic. The adherent HUH7 cells used to test the efficacy of AAV products were cultured under standard conditions at 37° C. and 5% CO₂, on a complete DMEM medium supplemented with 10% FBS, antibiotic/antimycotic. Cells were subcultured upon reaching 80-90% confluence. TrypLE Select enzyme (10x) was used to dissociate the cell monolayer. Cell viability was assessed using Trypan Blue stain and disposable cell counting chambers using an automatic Countess II counter.

### Transfection of Cell Cultures

To assess the functionality of the new codon-optimized nucleic acids that encode the FVIII-BDD protein, plasmids comprising an expression cassette for expressing various variants of hFVIII-BDD transgenes were used in transfection. SK-HEP1 cells were pre-seeded into wells of 12-well plates at a density of 10000 cells/cm². A day later, plasmids with the same copy number were introduced in a complex with Lipofectamine 3000. On day 7 following transfection, the level and activity of the FVIII-BDD protein in the culture fluid were determined by ELISA and chromogenic assay. Studies involving the assessment of the level and activity of the FVIII-BDD protein in the culture fluid were performed in 6 independent experiments. Intact SK-HEP1 cells were used as a negative control.

### Assembly and Purification of Expression Vectors Based on AAV

To assemble AAV expression vectors comprising variants of the FVIII-BDD gene, we used the HEK293 producer cells which were transfected with 3 plasmids as follows:
1) plasmids comprising an AAV expression cassette for expressing various variants of hFVIII-BDD transgenes (FIG. 1.);
2) a plasmid for expressing the AAV6 or AAV5 serotype Cap gene and the AAV2 serotype Rep gene. Each gene, using alternative reading frames, encodes several protein products;
3) a plasmid for expressing adenovirus Ad5 genes that are required for assembly and packaging of AAV capsids.

After 72 hours, the cells were lysed, and the vectors were purified and concentrated using filtration, chromatography and ultracentrifugation methods. The titer of the viral vectors was determined by quantitative PCR with primers and a sample that were specific for the region of the recombinant viral genome and expressed as the copy number of viral genomes per 1 ml.

### Transduction of Cell Cultures

The HUH7 cell line was pre-seeded into the wells of 12-well plates at a density of 10000 cells/cm². After the cells were attached to the adhesive substrate, AAV preparations were introduced at MOI of 500000 vg/cell. On day 7 following transduction, the level and activity of the FVIII-BDD protein in the culture fluid were determined by ELISA and chromogenic assay. Studies involving the assessment of the level and activity of the FVIII protein in the culture fluid were performed in 6 independent experiments. Intact cells were used as a negative control.

### Determination of Amount of Coagulation Factor VIII-BDD Protein by ELISA

Coagulation factor VIII-BDD protein levels in culture fluid following cell transfection with target candidates were assessed by sandwich method of non-competitive solid-phase enzyme immunoassay (ELISA). Briefly, culture fluid samples diluted in a dilution buffer were introduced into 96-well plate wells sensitized with primary antibodies specific for coagulation factor VIII-BDD. The same plate was loaded with standards for plotting a calibration curve, controls. The plate was incubated for 1 hour at a temperature of 37° C. The plate wells were washed with washing buffer prior to introducing biotinylated antibodies, solution of streptavidin peroxidase conjugate and TMB. A solution containing biotinylated detecting antibodies specific for factor VIII-BDD was introduced, and the plate was incubated for 30 minutes at a temperature of 37° C. Streptavidin peroxidase conjugate solution was then added to the resulting complex, and the plate was incubated for 30 minutes at a temperature of 37° C. TMB solution was introduced to visualize the enzyme reaction. Upon achieving the required degree of staining intensity, a stop solution was added to all wells to stop the reaction. The optical density of the solutions in the plate wells was then measured. The concentration of coagulation factor VIII-BDD in the test samples was determined by the calibration curve considering the preliminary dilution of the samples.

### Determination of Activity Level of Coagulation Factor VIII-BDD Protein by ELISA

The activity of the coagulation factor VIII protein in the culture fluid following transfection of the cells with target candidates was assessed by a chromogenic assay. The assay is based on the fact that in the presence of calcium ions, phospholipids and factor IXa, factor X transforms into the activated form Xa, factor VIII functions as a cofactor in the reaction, and the rate of factor X activation is linearly associated with the level of factor VIII. Briefly, culture fluid samples diluted in a dilution buffer, standards for plotting a calibration curve and controls were introduced into the wells of a 96-well plate. The plate was incubated for 3 minutes at a temperature of 37° C. A Factor reagent solution comprising factor IXa, factor X, thrombin, CaCl and phospholipids was introduced into all wells of the plate. The plate was incubated for 4 minutes at a temperature of 37° C. A solution of chromogenic substrate S-2765+1-2581 was introduced into all wells of the plate. The plate was incubated for 7 minutes at a temperature of 37° C. Upon achieving the required degree of staining intensity, a 20% solution of acetic acid was added to all wells to stop the reaction. The optical density of the solutions in the plate wells was then measured. The activity of coagulation factor VIII in the test samples was determined by the calibration curve considering the preliminary dilution of the samples.

### In Vivo Study on Laboratory Animals

The experiments were performed on C57BL/6 mice (males aged 6-8 weeks). The products were administered to animals by way of a single intravenous injection into the tail vein. A buffer solution free of AAV was adminitered into the negative control group of animals. Blood plasma sampling was performed on the day of injection before administering the products, then on days 35 and 56 following introducing the expression vectors.

### Statistical Data Analysis

The results indicate an average value±standard deviation (SD), one-way analysis of variance (ANOVA) followed by Dunnett's multiple pairwise comparisons was employed to compare the experiment results, and they were determined to be statistically significant.

### Example 1: Modification of hFVIII-BDD Gene Sequence Using Developed Codon Optimization Algorithm

The developed genetic constructs are nucleotide sequences encoding an expression cassette with a human B-domain deleted coagulation factor VIII gene (FVIII-BDD).

The resulting expression cassette comprises all the elements required for both gene expression and assembly in the recombinant AAV genome (FIG. 1):
1) ITRs at the ends of the sequence that is encapsidated into an expression vector;
2) elements for expressing the target gene (promoter, transgene, polyadenylation site);
3) bacterial replication origin and antibiotic resistance gene for producing plasmid DNA in bacterial cells.

As a wild-type nucleic acid, we used a nucleic acid that encodes the human B-domain deleted coagulation factor VIII protein, and includes the nucleotide sequence of SEQ ID NO: 1. The given nucleic acid is used within the expression cassette as a control.

Further, to increase the efficiency of expression, the naturally-occurring nucleotide sequence of the B-domain deleted coagulation factor VIII gene was modified using a codon optimization algorithm.

Codon optimization of the nucleic acid corresponding to the sequence of SEQ ID NO: 1 resulted in a number of codon-optimized nucleic acids of SEQ ID NO:2, SEQ ID NO:3, SEQ ID NO:4 or SEQ ID NO: 5 which were further tested for coagulation factor VIII-BDD protein expression and activity levels as compared to the control (nucleic acid of SEQ ID NO: 1) within the expression cassette (FIG. 1).

### Example 2. Functional Testing of Codon-Optimized Variants of B-Domain Deleted FVIII Gene Sequence In Vitro

The produced codon-optimized nucleic acids selected from SEQ ID NO:2, SEQ ID NO:3, SEQ ID NO: 4 or SEQ ID NO:5 encoding the FVIII-BDD protein were checked by transfecting SK-HEP1 cells in vitro as part of an expression cassette consisting of a left-hand (first) ITR (inverted terminal repeats), tissue-specific promoter, transgene, polyadenylation signal, right-hand (second) ITR, wherein the transgene is one of the sequences of SEQ ID NO: 2-5. As a control, the transgen was a non-optimized nucleic acid encoding the human B-domain deleted FVIII protein, the non-optimized nucleic acid corresponding to the sequence of SEQ ID NO:1.

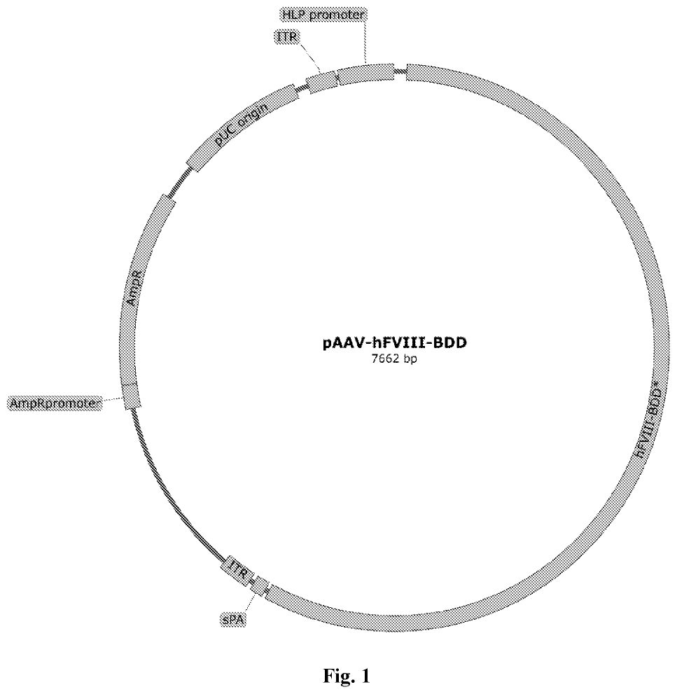

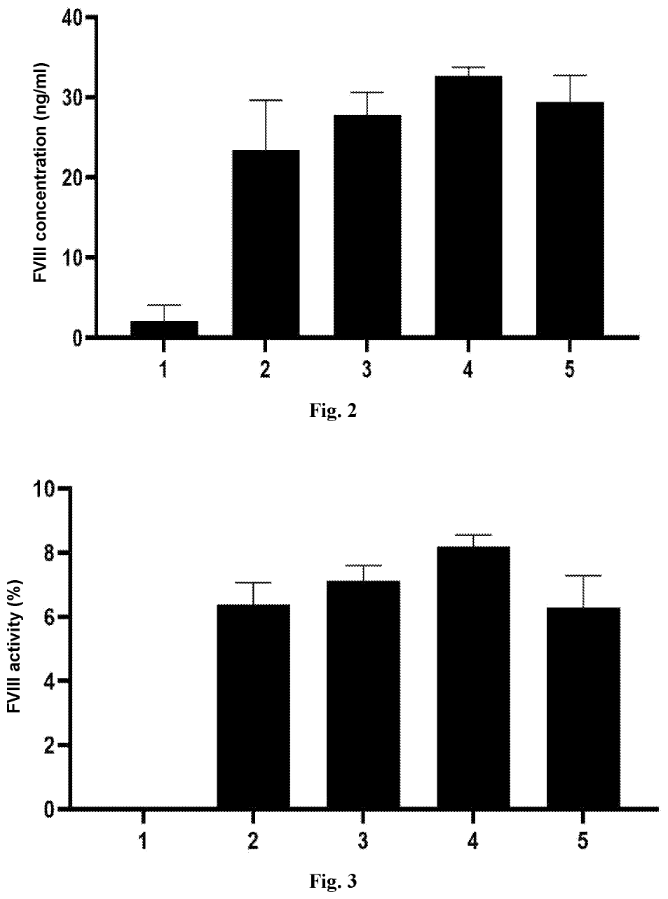

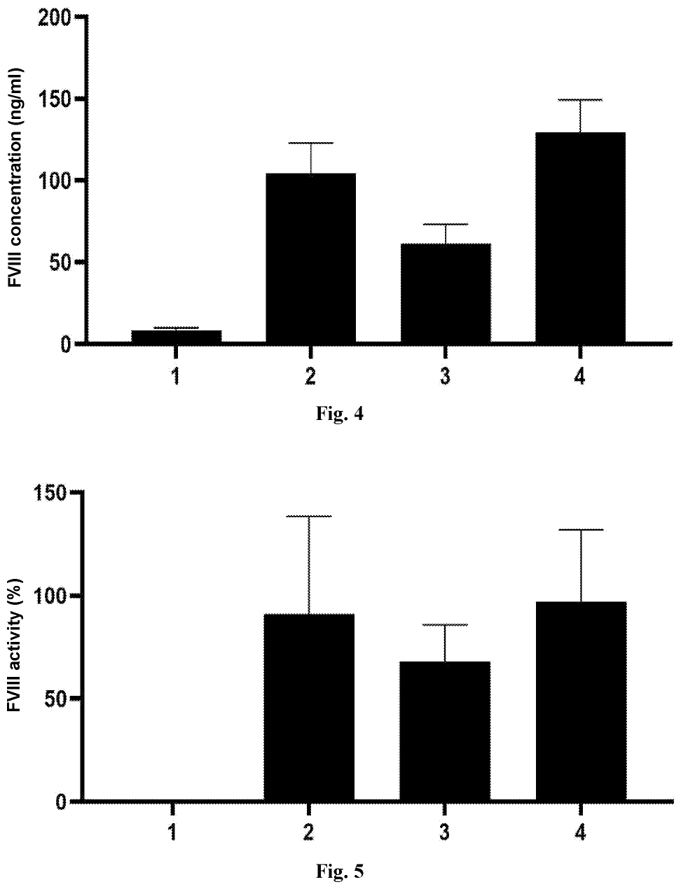

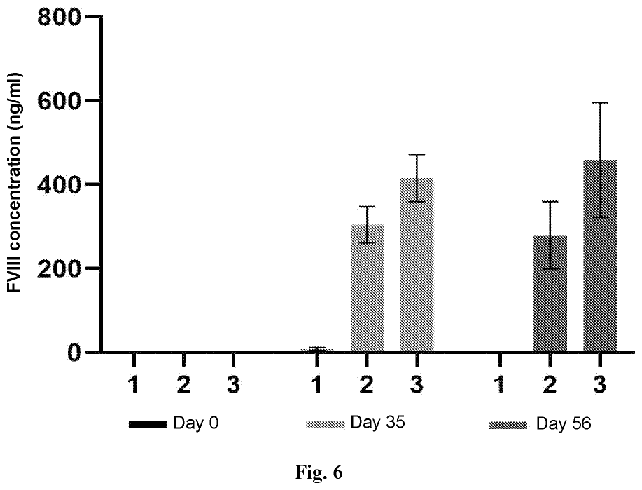

Use of all the nucleic acids selected from SEQ ID NO:2, SEQ ID NO:3, SEQ ID NO:4 or SEQ ID NO: 5 encoding the FVIII-BDD protein caused increased levels of production (FIG. 2) and activity (FIG. 3) of the FVIII-BDD protein as compared to the use of the non-optimized nucleic acid of SEQ ID NO:1.

Furthermore, the produced codon-optimized nucleic acids encoding the B-domain deleted coagulation factor VIII protein (SEQ ID NO:2, SEQ ID NO:3, SEQ ID NO:4 or SEQ ID NO:5) are capable of causing increased expression of the FVIII-BDD protein in vitro in the host cell as compared to the use of the non-optimized nucleic acid of SEQ ID NO: 1. Thus, the codon-optimized nucleic acids according to the invention have a high potential for producing a recombinant FVIII-BDD protein for the therapy of Haemophilia A.

### Example 3. Creation and Functional Testing of Expression Vectors Bearing Codon-Optimized Variants of FVIII-BDD Gene Sequence In Vitro

To prove the efficiency of delivery of the developed codon-optimized nucleic acids using an expression vector in vitro, we used plasmids encoding target genetic constructs together with the rest of the necessary plasmids to generate expression vectors based on adeno-associated virus serotype 5 bearing the nucleotide sequence of SEQ ID NO:2 (hereinafter referred to as AAV5-FVIII-BDDco-v1), and based on adeno-associated virus serotype 6 bearing the nucleotide sequence of SEQ ID NO:2 (hereinafter referred to as AAV6-FVIII-BDDco-v1) or the nucleotide sequence of SEQ ID NO:5 (hereinafter referred to as AAV6-FVIII-BDDco-v4). The purified AAV5-FVIII-BDDco-v1, AAV6-FVIII-BDDco-v1 and AAV6-FVIII-BDDco-v4 products used for in vitro and in vivo studies were prepared using standard buffers and excipients that are safe and do not alter AAV properties.

The AAV5-FVIII-BDDco-v1, AAV6-FVIII-BDDco-v1 and AAV6-FVIII-BDDco-v4 products were tested in vitro using adherent HUH7 cell line (FIGS. 4 and 5). The cell line's cells were plated into the wells of 12-well plates at a density of 10,000 cells/cm². After the cells were attached to the adhesive substrate, AAV preparations were introduced at MOI of 500,000 vg/cell. On day 7 following transduction, the level and activity of the FVIII-BDD protein in the culture fluid were determined by ELISA. All samples were run in triplicates. Intact cells were used as a negative control.

It was shown that the produced AAV5-FVIII-BDDco-v1, AAV6-FVIII-BDDco-v1 and AAV6-FVIII-BDDco-v4 expression vectors bearing codon-optimized variants of the coagulation factor VIII-BDD gene are capable of effectively delivering the coagulation factor VIII-BDD transgene to HUH7 cells and provide the production of the target protein, as confirmed by a culture fluid analysis by ELISA (FIG. 4) and activity analysis (FIG. 5) of the coagulation factor VIII-BDD protein. Delivery of SEQ ID NO:2 using the AAV serotype 5 expression vector causes the production of FVIII-BDD protein in culture fluid at 129 ng/ml and activity at 97%. Delivery of SEQ ID NO:2 using the AAV serotype 6 expression vector causes the production of FVIII-BDD protein in culture fluid at 104 ng/ml and activity at 91%. Delivery of SEQ ID NO:5 using the AAV serotype 6 expression vector causes the production of FVIII-BDD protein in culture fluid at 61 ng/ml and activity at 68%.

### Example 4. Functional Testing of AAV5-FVIII-BDDco-v1 and AAV6-FVIII-BDDco-v1 Products In Vivo

To show the efficiency of delivery of the developed codon-optimized nucleic acids when delivered within an expression vector in vivo, the selected products AAV5-FVIII-BDDco-v1 and AAV6-FVIII-BDDco-v1 were administered to laboratory C57BL/6 mice. The dose of AAV products used in the study was 6x1013 vg/mouse. A control solution free of AAV was used as a negative control. The products were administered to animals by way of a single intravenous hydrodynamic administration into the tail vein. Blood plasma sampling was performed on the day of injection before administering the products (day 0), then on days 35 and 56 following introducing the products. The level of the coagulation factor VIII-BDD protein in the blood plasma samples was determined by ELISA, as described above.

In vivo studies showed that when using the both products AAV5-FVIII-BDDco-v1 and AAV6-FVIII-BDDco-v1 comprising the coagulation factor VIII-BDD gene codon-optimized sequence of SEQ ID NO: 2, there are observed significantly increased levels of factor VIII-BDD in blood plasma of animals on days 35 and 56 (FIG. 6). The product AAV5-FVIII-BDDco-v1 comprising the coagulation factor VIII-BDD gene codon-optimized sequence of SEQ ID NO:2 resulted in FVIII-BDD expression at 304 and 279 ng/ml in blood plasma of animals on days 35 and 56, respectively, following injection. The product AAV6-FVIII-BDDco-v1 comprising the coagulation factor VIII-BDD gene codon-optimized sequence of SEQ ID NO: 2 resulting in FVIII-BDD expression at 415 and 459 ng/ml in blood plasma of animals on days 35 and 56, respectively, following injection.

Accordingly, the developed AAV5 and AAV6 expression vectors bearing codon-optimized versions of the B-domain deleted coagulation factor VIII gene (AAV5-FVIII-BDDco-v1 and AAV6-FVIII-BDDco-v1) are capable of causing expression of the FVIII-BDD protein in vivo and have high potential for gene therapy of Haemophilia A.

1. An isolated codon-optimized nucleic acid encoding the FVIII-BDD (B-domain deleted coagulation factor VIII) protein with the amino acid sequence of SEQ ID NO: 11, which includes a nucleotide sequence that is selected from the group: SEQ ID NO: 2, SEQ ID NO: 3, SEQ ID NO: 4 or SEQ ID NO: 5.

2. An expression cassette that includes the nucleic acid according to claim 1.

3. The expression cassette according to claim 2, comprising the following elements in the 5′-end to 3′-end direction:
a left-hand (first) ITR (inverted terminal repeats);
a promoter;
a nucleic acid encoding a signal peptide;
the nucleic acid according to claim 1;
a polyadenylation signal;
a right-hand (second) ITR.

4. An expression vector that includes the nucleic acid according to claim 1 or the expression cassette according to any one of claims 2 to 3.

5. The expression vector according to claim 4, wherein the expression vector is a recombinant adeno-associated virus (AAV).

6. The expression vector according to claim 5, wherein the AAV is selected from a group including the following AAV serotypes: AAV1, AAV2, AAV3, AAV4, AAV5, AAV6, AAV7, AAV8, AAV9, AAV10 AAV11, AAV12, AAV13, AAV14, AAV15, AAV16, rAAV.rh8, rAAV.rhlO, rAAV.rh20, rAAV.rh39, rAAV.Rh74, rAAV.RHM4-1, AAV.hu37, rAAV.Anc80, rAAV.Anc80L65, rAAV.7m8, rAAV.PHP.B, rAAV2.5, rAAV2tYF, rAAV3B, rAAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15 or AAV.HSC16.

7. A host cell for producing the FVIII-BDD protein or for producing the expression vector according to claims 4 to 6, comprising the nucleic acid according to claim 1.

8. A pharmaceutical composition for delivering the FVIII-BDD gene to target cells, comprising the expression vector according to any one of claims 4 to 6 or cassette according to any one of claims 2 to 3 in combination with one or more pharmaceutically acceptable excipients.

9. Use of the expression vector according to any one of claims 4 to 6 or the cassette according to any one of claims 2 to 3 or the composition according to claim 8 for delivering the FVIII-BDD gene to target cells.

10. Use of the expression vector according to any one of claims 4 to 6 or the cassette according to any one of claims 2 to 3 or the composition according to claim 8 for providing the FVIII-BDD protein to a subject who has haemophilia A and/or does not have functional copies of the FVIII gene.

11. A method of providing the FVIII-BDD protein to a subject with haemophilia A, comprising administering a therapeutically effective amount of the expression vector according to any one of claims 4 to 6 or the composition according to claim 8 into the cells of a subject in need thereof.

12. A method of delivering the FVIII-BDD gene to the target cells of a subject with haemophilia A, comprising administering the expression vector according to any one of claims 4 to 6 or the composition according to claim 8 into the cells of a subject.

13. The use of the expression vector according to any one of claims 4 to 6 or the composition according to claim 8 for treating haemophilia A in a subject who has haemophilia A.

14. A method of treating haemophilia A in a subject, comprising administering a therapeutically effective amount of the expression vector according to any one of claims 4 to 6 or the composition according to claim 8 to a subject who has haemophilia.


SEQUENCE LISTING (22 sequences)

SEQ ID NO: 1 - DNA, 4314 bp, synthetic construct
  source 1..4314: mol_type = other DNA
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 2 - DNA, 4314 bp, synthetic construct
  source 1..4314: mol_type = other DNA
gccaccagaaggtactacctgggcgccgtggagctgagctgggactacatgcagagcgacctgggcgagctgcctgtggacgccagattcccccccagagtgcctaagagcttccccttcaacaccagcgtggtgtacaagaagaccctgttcgtggagttcaccgaccacctgttcaacatcgccaagcccagacccccctggatgggcctgctgggccctaccatccaggccgaggtgtacgacaccgtggtgatcaccctgaagaacatggccagccaccccgtgagcctgcacgccgtgggcgtgagctactggaaggccagcgagggcgccgagtacgacgaccagaccagccagagagagaaggaggacgacaaggtgttccccggcggcagccacacctacgtgtggcaggtgctgaaggagaacggccccatggccagcgaccccctgtgcctgacctacagctacctgagccacgtggacctggtgaaggacctgaacagcggcctgatcggcgccctgctggtgtgcagagagggcagcctggccaaggagaagacccagaccctgcacaagttcatcctgctgttcgccgtgttcgacgagggcaagagctggcacagcgagaccaagaacagcctgatgcaggacagagacgccgccagcgccagagcctggcccaagatgcacaccgtgaacggctacgtgaacaggagcctgcccggcctgatcggctgccacagaaagagcgtgtactggcacgtgatcggcatgggcaccacccccgaggtgcacagcatcttcctggagggccacaccttcctggtgaggaaccacagacaggccagcctggagatcagccccatcaccttcctgaccgcccagaccctgctgatggacctgggccagttcctgctgttctgccacatcagcagccaccagcacgacggcatggaggcctacgtgaaggtggacagctgccccgaggagccccagctgagaatgaagaacaacgaggaggccgaggactacgacgacgacctgaccgacagcgagatggacgtggtgagattcgacgacgacaacagccccagcttcatccagatcaggagcgtggccaagaagcaccccaagacctgggtgcactacatcgccgccgaggaggaggactgggactacgcccccctggtgctggcccccgacgacagaagctacaagagccagtacctgaacaacggcccccagaggatcggcagaaagtacaagaaggtgcggttcatggcctacaccgacgagaccttcaagaccagggaggccatccagcacgagagcggcatcctgggccctctgctgtacggcgaggtgggcgacaccctgctgatcatcttcaagaaccaggccagcagaccctacaacatctacccccacggcatcaccgacgtgagacccctgtacagcagaagactgcccaagggcgtgaagcacctgaaggacttccccatcctgcccggcgagatcttcaagtacaagtggaccgtgaccgtggaggacggccccaccaagagcgaccccagatgcctgaccagatactacagcagcttcgtgaacatggagcgggacctggccagcggcctgatcggccccctgctgatctgctacaaggagagcgtggaccagagaggcaaccagatcatgagcgacaagcggaacgtgatcctgttcagcgtgttcgacgagaaccggagctggtacctgaccgagaacatccagaggttcctgcccaaccccgccggcgtgcagctggaggaccccgagttccaggccagcaacatcatgcacagcatcaacggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggacaccctgaccctgttccccttcagcggcgagaccgtgttcatgagcatggagaaccccggcctgtggatcctgggctgccacaacagcgacttcagaaacagaggcatgaccgccctgctgaaggtgagcagctgcgacaagaacaccggcgactactacgaggacagctacgaggacatcagcgcctacctgctgagcaagaacaacgccatcgagcccaggagcttcagccagaacccccccgtgctgaagagacaccagagagagatcaccaggaccaccctgcagagcgaccaggaggagatcgactacgacgacaccatcagcgtggagatgaagaaggaggacttcgacatctacgacgaggacgagaaccagagccccagaagcttccagaagaagaccaggcactacttcatcgccgccgtggagagactgtgggactacggcatgagcagcagcccccacgtgctgagaaacagagcccagagcggcagcgtgccccagttcaagaaggtggtgttccaggagttcaccgacggcagcttcacccagcccctgtacagaggcgagctgaacgagcacctgggcctgctgggcccctacatcagagccgaggtggaggacaacatcatggtgaccttccggaaccaggccagcagaccctacagcttctacagcagcctgatcagctacgaggaggaccagaggcagggcgccgagcccagaaagaacttcgtgaagcccaacgagaccaagacctacttctggaaggtgcagcaccacatggcccccaccaaggacgagttcgactgcaaggcctgggcctacttcagcgacgtggacctggagaaggacgtgcacagcggcctgatcggccccctgctggtgtgccacaccaacaccctgaaccccgcccacggcagacaggtgaccgtgcaggagttcgccctgttcttcaccatcttcgacgagaccaagagctggtacttcaccgagaacatggagcggaactgcagagccccctgcaacatccagatggaggaccccaccttcaaggagaactacaggttccacgccatcaacggctacatcatggacaccctgcccggcctggtgatggcccaggaccagagaatcagatggtacctgctgagcatgggcagcaacgagaacatccacagcatccacttcagcggccacgtgttcaccgtgaggaagaaggaggagtacaagatggccctgtacaacctgtaccccggcgtgttcgagaccgtggagatgctgcccagcaaggccggcatctggagagtggagtgcctgatcggcgagcacctgcacgccggcatgagcaccctgttcctggtgtacagcaacaagtgccagacccccctgggcatggccagcggccacatcagagacttccagatcaccgccagcggccagtacggccagtgggcccccaagctggccagactgcactacagcggcagcatcaacgcctggagcaccaaggagcccttcagctggatcaaggtggacctgctggcccccatgatcatccacggcatcaagacccagggcgccagacagaagttcagcagcctgtacatcagccagttcatcatcatgtacagcctggacggcaagaagtggcagacctacagaggcaacagcaccggcaccctgatggtgttcttcggcaacgtggacagcagcggcatcaagcacaacatcttcaacccccccatcatcgcccggtacatcaggctgcaccccacccactacagcatcaggagcaccctgagaatggagctgatgggctgcgacctgaacagctgcagcatgcccctgggcatggagagcaaggccatcagcgacgcccagatcaccgccagcagctacttcaccaacatgttcgccacctggagccccagcaaggccagactgcacctgcagggcagaagcaacgcctggagaccccaggtgaacaaccccaaggagtggctgcaggtggacttccagaagaccatgaaggtgaccggcgtgaccacccagggcgtgaagagcctgctgaccagcatgtacgtgaaggagttcctgatcagcagcagccaggacggccaccagtggaccctgttcttccagaacggcaaggtgaaggtgttccagggcaaccaggacagcttcacccccgtggtgaacagcctggacccccccctgctgaccagatacctgagaatccacccccagagctgggtgcaccagatcgccctgagaatggaggtgctgggctgcgaggcccaggacctgtac

SEQ ID NO: 3 - DNA, 4314 bp, synthetic construct
  source 1..4314: mol_type = other DNA
gccacaagaagatactatctgggcgccgtggagctgagctgggattatatgcagtccgacctgggcgagctgccagtggatgctagattccctcctagagtgcctaagagctttccattcaatacctccgtggtgtacaagaagaccctgttcgtggagtttaccgaccacctgttcaacatcgccaaacctagaccaccttggatgggactgctgggcccaactattcaggccgaagtgtacgataccgtggtgatcactctgaagaatatggcctcccaccctgtgagcctgcatgcagttggcgtgagctattggaaggccagcgagggagccgaatacgatgaccagaccagccagagggagaaggaagacgacaaggtgtttcccggaggaagccacacctatgtgtggcaggtgctgaaggagaatggacctatggccagcgatccactgtgcctgacctactcttatctgagccacgtggacctggtgaaggacctgaatagcggcctgattggcgcactgctggtgtgtagagagggatctctggccaaagagaagacccagaccctgcacaagtttatcctgctgtttgccgtgtttgacgagggcaaatcttggcacagcgagaccaaaaacagcctgatgcaggatagagacgcagcctctgctagagcttggcctaagatgcacaccgtgaatggctacgtgaacagatccctgcccggcctgattggatgtcacagaaagagcgtgtactggcacgtgatcggaatgggaactaccccagaagtgcacagcatctttctggagggacacacctttctggtgcggaatcatagacaggccagcctggaaatcagccctattacctttctgaccgcccagacactgctgatggatctgggacagtttctgctgttctgccacatcagctctcaccagcatgacggaatggaagcctacgtgaaggtggacagctgccctgaggaaccacagctgaggatgaagaataacgaggaggccgaggattacgacgatgacctgaccgattctgagatggacgtggtgagattcgacgacgataactctccctccttcatccagatcagatccgtggccaaaaagcaccctaaaacctgggtgcactatatcgccgcagaggaagaagactgggattacgcacctctggtgctggcccctgatgacagatcttataagtctcagtacctgaacaacggccctcagagaatcggcaggaagtacaagaaggtgagattcatggcctacacagacgagacattcaagaccagagaggccattcagcacgagtccggcattctgggaccactgctgtacggagaggtgggcgatacactgctgatcatcttcaaaaatcaggccagcaggccctacaacatctacccacatggcatcacagacgtgaggcctctgtacagcagaagactgcctaaaggcgtgaagcacctgaaggacttccccattctgccaggcgagattttcaagtacaagtggacagtgaccgtggaggatggacccacaaagtctgaccctagatgcctgaccaggtactatagctccttcgtgaacatggaaagggatctggcctctggactgattggcccactgctgatctgctacaaggaatccgtggaccagagaggaaaccagatcatgagcgataagaggaatgtgatcctgttcagcgtgttcgatgagaacaggagctggtatctgaccgagaacatccagagattcctgccaaatccagccggcgtgcagctggaagatcctgaatttcaggcctccaatatcatgcactccatcaatggctacgtgtttgacagcctgcagctgtctgtgtgtctgcacgaagtggcctactggtacatcctgagcattggagcccagaccgactttctgagcgtgtttttctctggctacaccttcaagcacaagatggtgtacgaggacaccctgacactgttcccattctcaggcgaaaccgtgtttatgagcatggagaacccaggactgtggattctgggctgccacaactccgacttcaggaatagaggaatgaccgccctgctgaaagtgagcagctgcgataagaacaccggcgattattacgaggactcctacgaggatatcagcgcctacctgctgtccaaaaacaatgccatcgagcccaggagcttctcccagaatccacctgtgctgaaaagacaccagagggagatcaccagaaccacactgcagtctgatcaggaggagatcgactatgacgacaccatcagcgtggagatgaagaaggaggactttgatatctacgacgaggatgagaaccagagccccaggagctttcagaagaagaccaggcactacttcatcgccgcagtggagagactgtgggactacggaatgtcttcctcccctcacgtgctgagaaatagagcccagtctggaagcgtgccacagtttaagaaggtggtgttccaggagtttaccgacggctctttcacccagccactgtatagaggcgaactgaacgaacacctgggcctgctgggaccttatatcagagccgaagtggaggacaacatcatggtgaccttcagaaaccaggccagcagaccttacagcttctactccagcctgatcagctacgaagaggatcagagacagggcgccgaacctaggaaaaattttgtgaagccaaacgagaccaagacctacttttggaaggtgcagcatcacatggcccccaccaaggatgagttcgattgtaaagcctgggcctacttctccgatgtggacctggaaaaggatgtgcacagcggactgatcggaccactgctggtgtgccacacaaataccctgaaccctgcccacggaagacaggtgacagtgcaggaatttgccctgttcttcaccatctttgacgagaccaagtcctggtacttcaccgagaacatggagagaaactgcagagccccttgcaacattcagatggaggacccaacattcaaggagaactacaggttccacgccatcaatggctacatcatggatacactgcctggcctggtgatggcccaggatcagagaattaggtggtacctgctgtctatgggctccaatgagaatatccactccatccacttcagcggccacgtgttcaccgtgagaaagaaagaggagtacaagatggccctgtacaacctgtacccaggcgtgtttgagaccgtggagatgctgccttctaaagctggcatctggagagtggagtgcctgatcggcgagcatctgcatgctggaatgtctaccctgtttctggtgtactccaacaagtgccagacacctctgggcatggcttctggacatatcagagactttcagatcaccgcctccggccagtacggacaatgggctccaaagctggccagactgcattactctggctctattaatgcctggagcaccaaggagcccttttcctggatcaaggtggatctgctggcccccatgattatccacggcatcaaaacccagggcgccaggcagaagttttcttccctgtacatcagccagttcatcatcatgtacagcctggatggcaagaagtggcagacctacagaggcaatagcaccggaaccctgatggtgtttttcggcaacgtggactcctccggcattaagcacaatatcttcaacccacctatcatcgccaggtacatcagactgcaccccacccactactctatcagaagcaccctgaggatggaactgatgggctgtgacctgaattcctgtagcatgcctctgggaatggagtctaaagccatcagtgacgcccagattaccgccagcagctactttaccaacatgtttgctacatggtccccctctaaggccagactgcacctgcagggaagatctaacgcctggagaccacaggtgaacaaccccaaagaatggctgcaggtggattttcagaagaccatgaaagtgaccggcgtgacaacacagggcgtgaaatctctgctgacctctatgtacgtgaaggagtttctgatcagcagcagccaggacggccatcagtggaccctgttttttcagaatggcaaggtgaaggtgttccagggcaatcaggactcattcactccagtggtgaacagcctggacccacctctgctgaccagatacctgagaattcacccacagtcctgggtgcatcagatcgccctgagaatggaagtgctgggatgtgaggcacaggacctgtac

SEQ ID NO: 4 - DNA, 4314 bp, synthetic construct
  source 1..4314: mol_type = other DNA
gccacaaggaggtactatctgggcgccgtggaactgagctgggactatatgcagagcgacctgggagagctgcctgtggatgccagatttccacctagggtgcccaagagcttccccttcaatacaagcgtggtgtacaagaagaccctgttcgtggagtttaccgaccacctgttcaacatcgccaagcctagacctccttggatgggactgctgggccctaccattcaggccgaagtgtacgacacagtggtgatcaccctgaagaatatggccagccatccagtgtccctgcacgcagtgggagtgtcttattggaaggcctctgagggcgccgagtacgatgatcagaccagccagagagagaaggaggacgacaaggtgttccccggaggaagccacacctacgtgtggcaggtgctgaaagagaatggcccaatggcctccgaccccctgtgtctgacatacagctacctgagccacgtggacctggtgaaggacctgaactctggcctgatcggagctctgctggtgtgtagagaaggcagcctggccaaggagaagacccagaccctgcacaagttcatcctgctgtttgccgtgttcgatgagggcaaaagctggcattccgagaccaagaattccctgatgcaggatagggacgctgccagcgctagagcctggcctaagatgcacactgtgaatggctacgtgaaccggagcctgccaggcctgatcggctgtcacagaaagagcgtgtactggcacgtgatcggcatgggcaccactcctgaggtgcactctatctttctggaaggccacacctttctggtgcggaaccatagacaggcctccctggaaatcagccccatcacctttctgaccgcccagaccctgctgatggatctgggacagttcctgctgttttgccacatctcctctcaccagcacgacggaatggaggcctacgtgaaagtggacagctgtcctgaagaacctcagctgcggatgaagaataacgaggaggccgaggactacgacgatgacctgaccgatagcgagatggacgtggtgagattcgacgacgataacagccccagcttcatccagatcagatccgtggccaagaagcaccccaagacatgggtgcactatatcgccgcagaggaggaggattgggactacgcacctctggtgctggctcctgacgatagatcctacaagtcccagtacctgaacaacggcccccagagaatcggcagaaagtacaagaaggtgagattcatggcctacaccgacgagacctttaagaccagagaggccatccagcacgagagcggcattctgggaccactgctgtatggagaggtgggcgatacactgctgatcatcttcaagaatcaggcctcccggccatacaacatctatccacacggcatcaccgacgtgagaccactgtactccaggagactgcccaagggcgtgaagcacctgaaggacttccctattctgccaggcgagatcttcaagtacaagtggacagtgaccgtggaagacggacctaccaaaagcgaccccagatgcctgacccggtactactccagcttcgtgaacatggagagagatctggccagcggactgattggacctctgctgatttgttacaaggagagcgtggaccagaggggcaaccagattatgagcgacaagcggaatgtgatcctgttctccgtgttcgacgagaaccgcagctggtatctgaccgagaatatccagaggttcctgccaaaccccgccggcgttcagctggaagatccagagtttcaggcctccaacatcatgcacagcatcaatggctacgtgttcgacagcctgcagctgtccgtgtgtctgcatgaagtggcctactggtatatcctgagcatcggcgcccagaccgattttctgtccgtgtttttctccggctacaccttcaagcacaagatggtgtacgaggacaccctgaccctgtttccttttagcggcgaaaccgtgttcatgagcatggagaacccaggactgtggattctgggctgtcacaacagcgacttcagaaatcggggcatgaccgctctgctgaaggtgagctcttgtgacaaaaataccggcgactactacgaggacagctacgaggatatcagcgcctacctgctgagcaagaacaacgccatcgagccaagaagcttcagccagaatccccccgtgctgaagagacatcagagggagatcaccagaaccaccctgcagagcgaccaggaggagatcgattacgacgacaccatcagcgtggagatgaagaaggaggatttcgacatctacgacgaggacgagaatcagagcccaaggagctttcagaagaagacccggcactacttcatcgctgccgtggagagactgtgggattatggcatgagctccagccctcacgtgctgagaaatagagcccagtctggctccgtgccccagttcaagaaagtggtgttccaggaattcaccgacggcagctttacccagcccctgtatagaggcgaactgaatgaacacctgggcctgctgggaccttacatcagagccgaagtggaggacaacatcatggtgacctttaggaatcaggccagcagaccctacagcttctatagctccctgatctcctacgaggaggaccagagacagggcgctgagccaagaaagaatttcgtgaagcccaacgagaccaagacctacttctggaaagtgcagcaccacatggcccctaccaaggatgagttcgactgtaaagcctgggcctacttcagcgacgtggacctggagaaggacgtgcactccggactgatcggaccactgctggtgtgccacacaaacaccctgaaccctgcccatggaagacaggtgactgtgcaggaattcgccctgttctttaccatcttcgacgagaccaagtcctggtacttcaccgagaatatggagaggaattgcagagccccctgtaacatccagatggaggaccccacctttaaggagaattacaggtttcacgccatcaacggctacatcatggacacactgccaggcctggtgatggcacaggatcagaggatcagatggtatctgctgagcatgggctccaacgagaacatccactccatccacttcagcggccacgtgtttaccgtgagaaagaaggaggagtacaagatggccctgtacaacctgtatcccggcgtgttcgaaaccgtggagatgctgccttccaaagccggaatctggagagtggagtgcctgattggcgagcacctgcacgctggaatgtccaccctgttcctggtgtatagcaacaagtgccagacccccctgggcatggctagcggacacattagagactttcagatcaccgcctccggccagtacggacagtgggccccaaaactggctaggctgcattacagcggctccatcaatgcctggtccaccaaggagccattcagctggatcaaagtggatctgctggcccccatgatcatccacggcattaagacacagggcgccaggcagaagttcagctctctgtacatctcccagttcatcatcatgtactccctggacggcaagaagtggcagacctacaggggcaatagcaccggcacactgatggtgtttttcggcaacgtggactcttccggcatcaagcacaacatctttaacccccctatcatcgccaggtacatcaggctgcaccccactcactacagcatcagatccaccctgagaatggagctgatgggatgcgacctgaacagctgcagcatgcccctgggcatggagagcaaggccatttctgacgcccagatcaccgccagcagctatttcaccaatatgttcgctacctggagccccagcaaggccagactgcatctgcagggcagaagcaacgcctggagaccacaggtgaacaatcctaaagagtggctgcaggtggacttccagaagaccatgaaggtgaccggcgtgacaacccagggcgtgaaatctctgctgacctccatgtacgtgaaggagttcctgatctcctccagccaggatggccatcagtggaccctgttcttccagaacggcaaggtgaaagtgttccagggcaaccaggacagctttacccctgtggtgaactctctggatccccccctgctgaccaggtacctgagaattcacccccagagctgggtgcaccagatcgccctgagaatggaagtgctgggatgcgaggcacaggacctgtac

SEQ ID NO: 5 - DNA, 4314 bp, synthetic construct
  source 1..4314: mol_type = other DNA
gctacccggaggtactacctgggcgctgtggagctgagctgggactacatgcagagcgatctgggcgagctgcccgtggatgccagatttcccccaagagtgcctaagtccttccccttcaacaccagcgtggtgtacaagaagaccctgttcgtggagttcaccgaccacctgttcaacatcgccaagccaaggcccccctggatgggactgctgggacctacaattcaggccgaggtgtacgataccgtggtgatcaccctgaagaacatggcctcccacccagtgtccctgcatgccgtgggcgtgagctactggaaggcttctgaaggcgccgagtacgacgaccagaccagccagagggagaaggaggacgacaaggtgttcccaggcggatctcacacctacgtgtggcaggtgctgaaggagaatggcccaatggccagcgacccactgtgtctgacctacagctacctgtcccacgtggacctggtgaaggacctgaactccggcctgatcggcgccctgctggtgtgtagagaaggatctctggccaaggagaagacccagaccctgcacaagttcatcctgctgttcgccgtgtttgacgagggcaagagctggcattccgagaccaagaacagcctgatgcaggacagagacgcagccagcgctagagcctggcccaagatgcacaccgtgaatggctacgtgaaccggtccctgccaggcctgatcggctgtcacagaaaaagcgtgtactggcacgtgatcggcatgggcaccacacccgaggtgcacagcatctttctggagggccacaccttcctggtgagaaaccacaggcaggcctccctggagatctcccccatcacctttctgaccgcccagaccctgctgatggatctgggccagttcctgctgttctgccacatcagctcccaccagcacgacggaatggaggcctacgtgaaggtggatagctgccccgaggagccccagctgagaatgaagaacaacgaggaggccgaggactacgacgatgacctgaccgattccgagatggacgtggtgcgcttcgacgacgataatagccccagcttcatccagatcagatccgtggccaagaagcacccaaagacctgggtgcactacatcgctgccgaggaggaggactgggattacgccccactggtgctggcccctgatgacagatcctacaagagccagtacctgaacaacggcccccagagaatcggccggaagtacaagaaggtgagattcatggcctacaccgacgagaccttcaagaccagggaagccatccagcacgagagcggcatcctgggaccactgctgtacggagaagtgggcgataccctgctgatcatcttcaagaaccaggcctcccggccctacaacatctacccccacggcatcaccgacgtgaggccactgtacagcaggaggctgcccaagggcgtgaagcacctgaaggacttccccatcctgcccggcgagatctttaagtacaagtggaccgtgaccgtggaggacggcccaaccaagagcgacccaagatgcctgaccaggtactactcctccttcgtgaacatggagcgggatctggccagcggactgattggccctctgctgatctgctacaaggagagcgtggaccagcgcggcaatcagatcatgagcgacaagagaaacgtgatcctgttcagcgtgttcgacgagaatcgcagctggtacctgaccgagaacatccagaggttcctgcccaacccagccggcgtgcagctggaagatcctgaatttcaggccagcaacatcatgcactccatcaacggctacgtgttcgactccctgcagctgagcgtgtgtctgcacgaagtggcctactggtacatcctgagcatcggcgcccagaccgacttcctgtccgtgttcttttccggctacaccttcaagcacaagatggtgtacgaggacaccctgaccctgttcccctttagcggcgagaccgtgttcatgagcatggagaacccaggcctgtggattctgggctgccacaacagcgacttccgcaataggggcatgaccgccctgctgaaagtgtccagctgcgataagaacaccggcgactactacgaggacagctacgaggacatctccgcctacctgctgagcaagaacaacgccatcgagcccagatccttcagccagaaccctccagtgctgaagaggcaccagcgcgagatcaccagaaccaccctgcagtctgatcaggaggagatcgactacgacgacaccatcagcgtggagatgaagaaggaggacttcgacatctacgacgaggacgagaaccagagccctaggtccttccagaagaagacccggcactacttcatcgccgcagtggagaggctgtgggattacggcatgagcagctccccacacgtgctgagaaatagggcccagagcggcagcgtgccccagttcaaaaaggtggtgttccaggagttcaccgacggctccttcacccagcccctgtacagaggcgagctgaatgagcacctgggcctgctgggaccctacatcagagcagaggtggaggacaacatcatggtgaccttcaggaaccaggccagcagaccctacagcttctacagcagcctgatcagctacgaggaggaccagagacagggagccgagccacgcaagaacttcgtgaagcccaacgagaccaagacctacttctggaaggtgcagcaccacatggcccccaccaaggacgagttcgattgtaaagcctgggcctacttcagcgacgtggacctggagaaggacgtgcacagcggcctgatcggacctctgctggtgtgtcataccaacaccctgaaccccgcccacggcagacaggtgactgtgcaggagtttgccctgttcttcaccatcttcgacgagaccaagagctggtacttcaccgagaacatggagagaaactgcagggccccatgcaacatccagatggaagaccccaccttcaaggagaactaccggttccacgccatcaacggctacatcatggataccctgcctggcctggtgatggcccaggatcagagaatcaggtggtacctgctgagcatgggctccaacgagaacatccacagcatccacttctccggccacgtgttcaccgtgaggaaaaaggaggagtacaagatggccctgtacaacctgtaccccggcgtgtttgagaccgtggagatgctgccatctaaggccggcatttggagagtggagtgcctgatcggcgagcacctgcacgccggaatgtctaccctgttcctggtgtacagcaacaagtgccagacccccctgggcatggccagcggacacatcagagacttccagatcaccgccagcggccagtacggacagtgggctcctaaactggcccgcctgcactatagcggcagcattaacgcctggtccaccaaggagccattcagctggatcaaggtggatctgctggcccccatgatcatccacggcatcaaaacacagggcgccagacagaagttctccagcctgtacatctcccagttcatcatcatgtactccctggacggcaagaagtggcagacctacagaggcaactccaccggcacactgatggtgttcttcggcaacgtggacagcagcggcatcaagcacaacatcttcaacccccccatcatcgcccggtacatcagactgcacccaacccactactccatcaggtccaccctgaggatggagctgatgggctgcgatctgaattcctgcagcatgcccctgggcatggagtccaaagccatctctgacgcccagatcaccgccagctcctacttcaccaacatgttcgccacttggagccccagcaaggccagactgcacctgcagggcagaagcaatgcctggagaccccaggtgaacaacccaaaggagtggctgcaggtggacttccagaagaccatgaaggtgaccggcgtgaccacacagggcgtgaaaagcctgctgaccagcatgtacgtgaaggagttcctgatcagctccagccaggatgggcaccagtggaccctgttctttcagaacggcaaggtgaaggtgttccagggcaatcaggactcattcacccctgtggtgaactctctggacccccccctgctgacccggtacctgagaatccacccacagtcttgggtgcaccagatcgccctgaggatggaagtgctgggctgcgaggctcaggatctgtac

SEQ ID NO: 6 - DNA, 4371 bp, synthetic construct
  source 1..4371: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 7 - DNA, 4371 bp, synthetic construct
  source 1..4371: mol_type = other DNA
atgcagatcgagctgagcacctgcttcttcctgtgcctgctgaggttctgcttcagcgccaccagaaggtactacctgggcgccgtggagctgagctgggactacatgcagagcgacctgggcgagctgcctgtggacgccagattcccccccagagtgcctaagagcttccccttcaacaccagcgtggtgtacaagaagaccctgttcgtggagttcaccgaccacctgttcaacatcgccaagcccagacccccctggatgggcctgctgggccctaccatccaggccgaggtgtacgacaccgtggtgatcaccctgaagaacatggccagccaccccgtgagcctgcacgccgtgggcgtgagctactggaaggccagcgagggcgccgagtacgacgaccagaccagccagagagagaaggaggacgacaaggtgttccccggcggcagccacacctacgtgtggcaggtgctgaaggagaacggccccatggccagcgaccccctgtgcctgacctacagctacctgagccacgtggacctggtgaaggacctgaacagcggcctgatcggcgccctgctggtgtgcagagagggcagcctggccaaggagaagacccagaccctgcacaagttcatcctgctgttcgccgtgttcgacgagggcaagagctggcacagcgagaccaagaacagcctgatgcaggacagagacgccgccagcgccagagcctggcccaagatgcacaccgtgaacggctacgtgaacaggagcctgcccggcctgatcggctgccacagaaagagcgtgtactggcacgtgatcggcatgggcaccacccccgaggtgcacagcatcttcctggagggccacaccttcctggtgaggaaccacagacaggccagcctggagatcagccccatcaccttcctgaccgcccagaccctgctgatggacctgggccagttcctgctgttctgccacatcagcagccaccagcacgacggcatggaggcctacgtgaaggtggacagctgccccgaggagccccagctgagaatgaagaacaacgaggaggccgaggactacgacgacgacctgaccgacagcgagatggacgtggtgagattcgacgacgacaacagccccagcttcatccagatcaggagcgtggccaagaagcaccccaagacctgggtgcactacatcgccgccgaggaggaggactgggactacgcccccctggtgctggcccccgacgacagaagctacaagagccagtacctgaacaacggcccccagaggatcggcagaaagtacaagaaggtgcggttcatggcctacaccgacgagaccttcaagaccagggaggccatccagcacgagagcggcatcctgggccctctgctgtacggcgaggtgggcgacaccctgctgatcatcttcaagaaccaggccagcagaccctacaacatctacccccacggcatcaccgacgtgagacccctgtacagcagaagactgcccaagggcgtgaagcacctgaaggacttccccatcctgcccggcgagatcttcaagtacaagtggaccgtgaccgtggaggacggccccaccaagagcgaccccagatgcctgaccagatactacagcagcttcgtgaacatggagcgggacctggccagcggcctgatcggccccctgctgatctgctacaaggagagcgtggaccagagaggcaaccagatcatgagcgacaagcggaacgtgatcctgttcagcgtgttcgacgagaaccggagctggtacctgaccgagaacatccagaggttcctgcccaaccccgccggcgtgcagctggaggaccccgagttccaggccagcaacatcatgcacagcatcaacggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggacaccctgaccctgttccccttcagcggcgagaccgtgttcatgagcatggagaaccccggcctgtggatcctgggctgccacaacagcgacttcagaaacagaggcatgaccgccctgctgaaggtgagcagctgcgacaagaacaccggcgactactacgaggacagctacgaggacatcagcgcctacctgctgagcaagaacaacgccatcgagcccaggagcttcagccagaacccccccgtgctgaagagacaccagagagagatcaccaggaccaccctgcagagcgaccaggaggagatcgactacgacgacaccatcagcgtggagatgaagaaggaggacttcgacatctacgacgaggacgagaaccagagccccagaagcttccagaagaagaccaggcactacttcatcgccgccgtggagagactgtgggactacggcatgagcagcagcccccacgtgctgagaaacagagcccagagcggcagcgtgccccagttcaagaaggtggtgttccaggagttcaccgacggcagcttcacccagcccctgtacagaggcgagctgaacgagcacctgggcctgctgggcccctacatcagagccgaggtggaggacaacatcatggtgaccttccggaaccaggccagcagaccctacagcttctacagcagcctgatcagctacgaggaggaccagaggcagggcgccgagcccagaaagaacttcgtgaagcccaacgagaccaagacctacttctggaaggtgcagcaccacatggcccccaccaaggacgagttcgactgcaaggcctgggcctacttcagcgacgtggacctggagaaggacgtgcacagcggcctgatcggccccctgctggtgtgccacaccaacaccctgaaccccgcccacggcagacaggtgaccgtgcaggagttcgccctgttcttcaccatcttcgacgagaccaagagctggtacttcaccgagaacatggagcggaactgcagagccccctgcaacatccagatggaggaccccaccttcaaggagaactacaggttccacgccatcaacggctacatcatggacaccctgcccggcctggtgatggcccaggaccagagaatcagatggtacctgctgagcatgggcagcaacgagaacatccacagcatccacttcagcggccacgtgttcaccgtgaggaagaaggaggagtacaagatggccctgtacaacctgtaccccggcgtgttcgagaccgtggagatgctgcccagcaaggccggcatctggagagtggagtgcctgatcggcgagcacctgcacgccggcatgagcaccctgttcctggtgtacagcaacaagtgccagacccccctgggcatggccagcggccacatcagagacttccagatcaccgccagcggccagtacggccagtgggcccccaagctggccagactgcactacagcggcagcatcaacgcctggagcaccaaggagcccttcagctggatcaaggtggacctgctggcccccatgatcatccacggcatcaagacccagggcgccagacagaagttcagcagcctgtacatcagccagttcatcatcatgtacagcctggacggcaagaagtggcagacctacagaggcaacagcaccggcaccctgatggtgttcttcggcaacgtggacagcagcggcatcaagcacaacatcttcaacccccccatcatcgcccggtacatcaggctgcaccccacccactacagcatcaggagcaccctgagaatggagctgatgggctgcgacctgaacagctgcagcatgcccctgggcatggagagcaaggccatcagcgacgcccagatcaccgccagcagctacttcaccaacatgttcgccacctggagccccagcaaggccagactgcacctgcagggcagaagcaacgcctggagaccccaggtgaacaaccccaaggagtggctgcaggtggacttccagaagaccatgaaggtgaccggcgtgaccacccagggcgtgaagagcctgctgaccagcatgtacgtgaaggagttcctgatcagcagcagccaggacggccaccagtggaccctgttcttccagaacggcaaggtgaaggtgttccagggcaaccaggacagcttcacccccgtggtgaacagcctggacccccccctgctgaccagatacctgagaatccacccccagagctgggtgcaccagatcgccctgagaatggaggtgctgggctgcgaggcccaggacctgtac

SEQ ID NO: 8 - DNA, 4371 bp, synthetic construct
  source 1..4371: mol_type = other DNA
atgcagatcgagctgagcacctgtttttttctgtgcctgctgaggttctgcttctccgccacaagaagatactatctgggcgccgtggagctgagctgggattatatgcagtccgacctgggcgagctgccagtggatgctagattccctcctagagtgcctaagagctttccattcaatacctccgtggtgtacaagaagaccctgttcgtggagtttaccgaccacctgttcaacatcgccaaacctagaccaccttggatgggactgctgggcccaactattcaggccgaagtgtacgataccgtggtgatcactctgaagaatatggcctcccaccctgtgagcctgcatgcagttggcgtgagctattggaaggccagcgagggagccgaatacgatgaccagaccagccagagggagaaggaagacgacaaggtgtttcccggaggaagccacacctatgtgtggcaggtgctgaaggagaatggacctatggccagcgatccactgtgcctgacctactcttatctgagccacgtggacctggtgaaggacctgaatagcggcctgattggcgcactgctggtgtgtagagagggatctctggccaaagagaagacccagaccctgcacaagtttatcctgctgtttgccgtgtttgacgagggcaaatcttggcacagcgagaccaaaaacagcctgatgcaggatagagacgcagcctctgctagagcttggcctaagatgcacaccgtgaatggctacgtgaacagatccctgcccggcctgattggatgtcacagaaagagcgtgtactggcacgtgatcggaatgggaactaccccagaagtgcacagcatctttctggagggacacacctttctggtgcggaatcatagacaggccagcctggaaatcagccctattacctttctgaccgcccagacactgctgatggatctgggacagtttctgctgttctgccacatcagctctcaccagcatgacggaatggaagcctacgtgaaggtggacagctgccctgaggaaccacagctgaggatgaagaataacgaggaggccgaggattacgacgatgacctgaccgattctgagatggacgtggtgagattcgacgacgataactctccctccttcatccagatcagatccgtggccaaaaagcaccctaaaacctgggtgcactatatcgccgcagaggaagaagactgggattacgcacctctggtgctggcccctgatgacagatcttataagtctcagtacctgaacaacggccctcagagaatcggcaggaagtacaagaaggtgagattcatggcctacacagacgagacattcaagaccagagaggccattcagcacgagtccggcattctgggaccactgctgtacggagaggtgggcgatacactgctgatcatcttcaaaaatcaggccagcaggccctacaacatctacccacatggcatcacagacgtgaggcctctgtacagcagaagactgcctaaaggcgtgaagcacctgaaggacttccccattctgccaggcgagattttcaagtacaagtggacagtgaccgtggaggatggacccacaaagtctgaccctagatgcctgaccaggtactatagctccttcgtgaacatggaaagggatctggcctctggactgattggcccactgctgatctgctacaaggaatccgtggaccagagaggaaaccagatcatgagcgataagaggaatgtgatcctgttcagcgtgttcgatgagaacaggagctggtatctgaccgagaacatccagagattcctgccaaatccagccggcgtgcagctggaagatcctgaatttcaggcctccaatatcatgcactccatcaatggctacgtgtttgacagcctgcagctgtctgtgtgtctgcacgaagtggcctactggtacatcctgagcattggagcccagaccgactttctgagcgtgtttttctctggctacaccttcaagcacaagatggtgtacgaggacaccctgacactgttcccattctcaggcgaaaccgtgtttatgagcatggagaacccaggactgtggattctgggctgccacaactccgacttcaggaatagaggaatgaccgccctgctgaaagtgagcagctgcgataagaacaccggcgattattacgaggactcctacgaggatatcagcgcctacctgctgtccaaaaacaatgccatcgagcccaggagcttctcccagaatccacctgtgctgaaaagacaccagagggagatcaccagaaccacactgcagtctgatcaggaggagatcgactatgacgacaccatcagcgtggagatgaagaaggaggactttgatatctacgacgaggatgagaaccagagccccaggagctttcagaagaagaccaggcactacttcatcgccgcagtggagagactgtgggactacggaatgtcttcctcccctcacgtgctgagaaatagagcccagtctggaagcgtgccacagtttaagaaggtggtgttccaggagtttaccgacggctctttcacccagccactgtatagaggcgaactgaacgaacacctgggcctgctgggaccttatatcagagccgaagtggaggacaacatcatggtgaccttcagaaaccaggccagcagaccttacagcttctactccagcctgatcagctacgaagaggatcagagacagggcgccgaacctaggaaaaattttgtgaagccaaacgagaccaagacctacttttggaaggtgcagcatcacatggcccccaccaaggatgagttcgattgtaaagcctgggcctacttctccgatgtggacctggaaaaggatgtgcacagcggactgatcggaccactgctggtgtgccacacaaataccctgaaccctgcccacggaagacaggtgacagtgcaggaatttgccctgttcttcaccatctttgacgagaccaagtcctggtacttcaccgagaacatggagagaaactgcagagccccttgcaacattcagatggaggacccaacattcaaggagaactacaggttccacgccatcaatggctacatcatggatacactgcctggcctggtgatggcccaggatcagagaattaggtggtacctgctgtctatgggctccaatgagaatatccactccatccacttcagcggccacgtgttcaccgtgagaaagaaagaggagtacaagatggccctgtacaacctgtacccaggcgtgtttgagaccgtggagatgctgccttctaaagctggcatctggagagtggagtgcctgatcggcgagcatctgcatgctggaatgtctaccctgtttctggtgtactccaacaagtgccagacacctctgggcatggcttctggacatatcagagactttcagatcaccgcctccggccagtacggacaatgggctccaaagctggccagactgcattactctggctctattaatgcctggagcaccaaggagcccttttcctggatcaaggtggatctgctggcccccatgattatccacggcatcaaaacccagggcgccaggcagaagttttcttccctgtacatcagccagttcatcatcatgtacagcctggatggcaagaagtggcagacctacagaggcaatagcaccggaaccctgatggtgtttttcggcaacgtggactcctccggcattaagcacaatatcttcaacccacctatcatcgccaggtacatcagactgcaccccacccactactctatcagaagcaccctgaggatggaactgatgggctgtgacctgaattcctgtagcatgcctctgggaatggagtctaaagccatcagtgacgcccagattaccgccagcagctactttaccaacatgtttgctacatggtccccctctaaggccagactgcacctgcagggaagatctaacgcctggagaccacaggtgaacaaccccaaagaatggctgcaggtggattttcagaagaccatgaaagtgaccggcgtgacaacacagggcgtgaaatctctgctgacctctatgtacgtgaaggagtttctgatcagcagcagccaggacggccatcagtggaccctgttttttcagaatggcaaggtgaaggtgttccagggcaatcaggactcattcactccagtggtgaacagcctggacccacctctgctgaccagatacctgagaattcacccacagtcctgggtgcatcagatcgccctgagaatggaagtgctgggatgtgaggcacaggacctgtac

SEQ ID NO: 9 - DNA, 4371 bp, synthetic construct
  source 1..4371: mol_type = other DNA
atgcagatcgagctgagcacctgtttcttcctgtgcctgctgaggttctgtttcagcgccacaaggaggtactatctgggcgccgtggaactgagctgggactatatgcagagcgacctgggagagctgcctgtggatgccagatttccacctagggtgcccaagagcttccccttcaatacaagcgtggtgtacaagaagaccctgttcgtggagtttaccgaccacctgttcaacatcgccaagcctagacctccttggatgggactgctgggccctaccattcaggccgaagtgtacgacacagtggtgatcaccctgaagaatatggccagccatccagtgtccctgcacgcagtgggagtgtcttattggaaggcctctgagggcgccgagtacgatgatcagaccagccagagagagaaggaggacgacaaggtgttccccggaggaagccacacctacgtgtggcaggtgctgaaagagaatggcccaatggcctccgaccccctgtgtctgacatacagctacctgagccacgtggacctggtgaaggacctgaactctggcctgatcggagctctgctggtgtgtagagaaggcagcctggccaaggagaagacccagaccctgcacaagttcatcctgctgtttgccgtgttcgatgagggcaaaagctggcattccgagaccaagaattccctgatgcaggatagggacgctgccagcgctagagcctggcctaagatgcacactgtgaatggctacgtgaaccggagcctgccaggcctgatcggctgtcacagaaagagcgtgtactggcacgtgatcggcatgggcaccactcctgaggtgcactctatctttctggaaggccacacctttctggtgcggaaccatagacaggcctccctggaaatcagccccatcacctttctgaccgcccagaccctgctgatggatctgggacagttcctgctgttttgccacatctcctctcaccagcacgacggaatggaggcctacgtgaaagtggacagctgtcctgaagaacctcagctgcggatgaagaataacgaggaggccgaggactacgacgatgacctgaccgatagcgagatggacgtggtgagattcgacgacgataacagccccagcttcatccagatcagatccgtggccaagaagcaccccaagacatgggtgcactatatcgccgcagaggaggaggattgggactacgcacctctggtgctggctcctgacgatagatcctacaagtcccagtacctgaacaacggcccccagagaatcggcagaaagtacaagaaggtgagattcatggcctacaccgacgagacctttaagaccagagaggccatccagcacgagagcggcattctgggaccactgctgtatggagaggtgggcgatacactgctgatcatcttcaagaatcaggcctcccggccatacaacatctatccacacggcatcaccgacgtgagaccactgtactccaggagactgcccaagggcgtgaagcacctgaaggacttccctattctgccaggcgagatcttcaagtacaagtggacagtgaccgtggaagacggacctaccaaaagcgaccccagatgcctgacccggtactactccagcttcgtgaacatggagagagatctggccagcggactgattggacctctgctgatttgttacaaggagagcgtggaccagaggggcaaccagattatgagcgacaagcggaatgtgatcctgttctccgtgttcgacgagaaccgcagctggtatctgaccgagaatatccagaggttcctgccaaaccccgccggcgttcagctggaagatccagagtttcaggcctccaacatcatgcacagcatcaatggctacgtgttcgacagcctgcagctgtccgtgtgtctgcatgaagtggcctactggtatatcctgagcatcggcgcccagaccgattttctgtccgtgtttttctccggctacaccttcaagcacaagatggtgtacgaggacaccctgaccctgtttccttttagcggcgaaaccgtgttcatgagcatggagaacccaggactgtggattctgggctgtcacaacagcgacttcagaaatcggggcatgaccgctctgctgaaggtgagctcttgtgacaaaaataccggcgactactacgaggacagctacgaggatatcagcgcctacctgctgagcaagaacaacgccatcgagccaagaagcttcagccagaatccccccgtgctgaagagacatcagagggagatcaccagaaccaccctgcagagcgaccaggaggagatcgattacgacgacaccatcagcgtggagatgaagaaggaggatttcgacatctacgacgaggacgagaatcagagcccaaggagctttcagaagaagacccggcactacttcatcgctgccgtggagagactgtgggattatggcatgagctccagccctcacgtgctgagaaatagagcccagtctggctccgtgccccagttcaagaaagtggtgttccaggaattcaccgacggcagctttacccagcccctgtatagaggcgaactgaatgaacacctgggcctgctgggaccttacatcagagccgaagtggaggacaacatcatggtgacctttaggaatcaggccagcagaccctacagcttctatagctccctgatctcctacgaggaggaccagagacagggcgctgagccaagaaagaatttcgtgaagcccaacgagaccaagacctacttctggaaagtgcagcaccacatggcccctaccaaggatgagttcgactgtaaagcctgggcctacttcagcgacgtggacctggagaaggacgtgcactccggactgatcggaccactgctggtgtgccacacaaacaccctgaaccctgcccatggaagacaggtgactgtgcaggaattcgccctgttctttaccatcttcgacgagaccaagtcctggtacttcaccgagaatatggagaggaattgcagagccccctgtaacatccagatggaggaccccacctttaaggagaattacaggtttcacgccatcaacggctacatcatggacacactgccaggcctggtgatggcacaggatcagaggatcagatggtatctgctgagcatgggctccaacgagaacatccactccatccacttcagcggccacgtgtttaccgtgagaaagaaggaggagtacaagatggccctgtacaacctgtatcccggcgtgttcgaaaccgtggagatgctgccttccaaagccggaatctggagagtggagtgcctgattggcgagcacctgcacgctggaatgtccaccctgttcctggtgtatagcaacaagtgccagacccccctgggcatggctagcggacacattagagactttcagatcaccgcctccggccagtacggacagtgggccccaaaactggctaggctgcattacagcggctccatcaatgcctggtccaccaaggagccattcagctggatcaaagtggatctgctggcccccatgatcatccacggcattaagacacagggcgccaggcagaagttcagctctctgtacatctcccagttcatcatcatgtactccctggacggcaagaagtggcagacctacaggggcaatagcaccggcacactgatggtgtttttcggcaacgtggactcttccggcatcaagcacaacatctttaacccccctatcatcgccaggtacatcaggctgcaccccactcactacagcatcagatccaccctgagaatggagctgatgggatgcgacctgaacagctgcagcatgcccctgggcatggagagcaaggccatttctgacgcccagatcaccgccagcagctatttcaccaatatgttcgctacctggagccccagcaaggccagactgcatctgcagggcagaagcaacgcctggagaccacaggtgaacaatcctaaagagtggctgcaggtggacttccagaagaccatgaaggtgaccggcgtgacaacccagggcgtgaaatctctgctgacctccatgtacgtgaaggagttcctgatctcctccagccaggatggccatcagtggaccctgttcttccagaacggcaaggtgaaagtgttccagggcaaccaggacagctttacccctgtggtgaactctctggatccccccctgctgaccaggtacctgagaattcacccccagagctgggtgcaccagatcgccctgagaatggaagtgctgggatgcgaggcacaggacctgtac

SEQ ID NO: 10 - DNA, 4371 bp, synthetic construct
  source 1..4371: mol_type = other DNA
atgcagatcgagctgtccacctgcttcttcctgtgcctgctgcgcttctgtttctctgctacccggaggtactacctgggcgctgtggagctgagctgggactacatgcagagcgatctgggcgagctgcccgtggatgccagatttcccccaagagtgcctaagtccttccccttcaacaccagcgtggtgtacaagaagaccctgttcgtggagttcaccgaccacctgttcaacatcgccaagccaaggcccccctggatgggactgctgggacctacaattcaggccgaggtgtacgataccgtggtgatcaccctgaagaacatggcctcccacccagtgtccctgcatgccgtgggcgtgagctactggaaggcttctgaaggcgccgagtacgacgaccagaccagccagagggagaaggaggacgacaaggtgttcccaggcggatctcacacctacgtgtggcaggtgctgaaggagaatggcccaatggccagcgacccactgtgtctgacctacagctacctgtcccacgtggacctggtgaaggacctgaactccggcctgatcggcgccctgctggtgtgtagagaaggatctctggccaaggagaagacccagaccctgcacaagttcatcctgctgttcgccgtgtttgacgagggcaagagctggcattccgagaccaagaacagcctgatgcaggacagagacgcagccagcgctagagcctggcccaagatgcacaccgtgaatggctacgtgaaccggtccctgccaggcctgatcggctgtcacagaaaaagcgtgtactggcacgtgatcggcatgggcaccacacccgaggtgcacagcatctttctggagggccacaccttcctggtgagaaaccacaggcaggcctccctggagatctcccccatcacctttctgaccgcccagaccctgctgatggatctgggccagttcctgctgttctgccacatcagctcccaccagcacgacggaatggaggcctacgtgaaggtggatagctgccccgaggagccccagctgagaatgaagaacaacgaggaggccgaggactacgacgatgacctgaccgattccgagatggacgtggtgcgcttcgacgacgataatagccccagcttcatccagatcagatccgtggccaagaagcacccaaagacctgggtgcactacatcgctgccgaggaggaggactgggattacgccccactggtgctggcccctgatgacagatcctacaagagccagtacctgaacaacggcccccagagaatcggccggaagtacaagaaggtgagattcatggcctacaccgacgagaccttcaagaccagggaagccatccagcacgagagcggcatcctgggaccactgctgtacggagaagtgggcgataccctgctgatcatcttcaagaaccaggcctcccggccctacaacatctacccccacggcatcaccgacgtgaggccactgtacagcaggaggctgcccaagggcgtgaagcacctgaaggacttccccatcctgcccggcgagatctttaagtacaagtggaccgtgaccgtggaggacggcccaaccaagagcgacccaagatgcctgaccaggtactactcctccttcgtgaacatggagcgggatctggccagcggactgattggccctctgctgatctgctacaaggagagcgtggaccagcgcggcaatcagatcatgagcgacaagagaaacgtgatcctgttcagcgtgttcgacgagaatcgcagctggtacctgaccgagaacatccagaggttcctgcccaacccagccggcgtgcagctggaagatcctgaatttcaggccagcaacatcatgcactccatcaacggctacgtgttcgactccctgcagctgagcgtgtgtctgcacgaagtggcctactggtacatcctgagcatcggcgcccagaccgacttcctgtccgtgttcttttccggctacaccttcaagcacaagatggtgtacgaggacaccctgaccctgttcccctttagcggcgagaccgtgttcatgagcatggagaacccaggcctgtggattctgggctgccacaacagcgacttccgcaataggggcatgaccgccctgctgaaagtgtccagctgcgataagaacaccggcgactactacgaggacagctacgaggacatctccgcctacctgctgagcaagaacaacgccatcgagcccagatccttcagccagaaccctccagtgctgaagaggcaccagcgcgagatcaccagaaccaccctgcagtctgatcaggaggagatcgactacgacgacaccatcagcgtggagatgaagaaggaggacttcgacatctacgacgaggacgagaaccagagccctaggtccttccagaagaagacccggcactacttcatcgccgcagtggagaggctgtgggattacggcatgagcagctccccacacgtgctgagaaatagggcccagagcggcagcgtgccccagttcaaaaaggtggtgttccaggagttcaccgacggctccttcacccagcccctgtacagaggcgagctgaatgagcacctgggcctgctgggaccctacatcagagcagaggtggaggacaacatcatggtgaccttcaggaaccaggccagcagaccctacagcttctacagcagcctgatcagctacgaggaggaccagagacagggagccgagccacgcaagaacttcgtgaagcccaacgagaccaagacctacttctggaaggtgcagcaccacatggcccccaccaaggacgagttcgattgtaaagcctgggcctacttcagcgacgtggacctggagaaggacgtgcacagcggcctgatcggacctctgctggtgtgtcataccaacaccctgaaccccgcccacggcagacaggtgactgtgcaggagtttgccctgttcttcaccatcttcgacgagaccaagagctggtacttcaccgagaacatggagagaaactgcagggccccatgcaacatccagatggaagaccccaccttcaaggagaactaccggttccacgccatcaacggctacatcatggataccctgcctggcctggtgatggcccaggatcagagaatcaggtggtacctgctgagcatgggctccaacgagaacatccacagcatccacttctccggccacgtgttcaccgtgaggaaaaaggaggagtacaagatggccctgtacaacctgtaccccggcgtgtttgagaccgtggagatgctgccatctaaggccggcatttggagagtggagtgcctgatcggcgagcacctgcacgccggaatgtctaccctgttcctggtgtacagcaacaagtgccagacccccctgggcatggccagcggacacatcagagacttccagatcaccgccagcggccagtacggacagtgggctcctaaactggcccgcctgcactatagcggcagcattaacgcctggtccaccaaggagccattcagctggatcaaggtggatctgctggcccccatgatcatccacggcatcaaaacacagggcgccagacagaagttctccagcctgtacatctcccagttcatcatcatgtactccctggacggcaagaagtggcagacctacagaggcaactccaccggcacactgatggtgttcttcggcaacgtggacagcagcggcatcaagcacaacatcttcaacccccccatcatcgcccggtacatcagactgcacccaacccactactccatcaggtccaccctgaggatggagctgatgggctgcgatctgaattcctgcagcatgcccctgggcatggagtccaaagccatctctgacgcccagatcaccgccagctcctacttcaccaacatgttcgccacttggagccccagcaaggccagactgcacctgcagggcagaagcaatgcctggagaccccaggtgaacaacccaaaggagtggctgcaggtggacttccagaagaccatgaaggtgaccggcgtgaccacacagggcgtgaaaagcctgctgaccagcatgtacgtgaaggagttcctgatcagctccagccaggatgggcaccagtggaccctgttctttcagaacggcaaggtgaaggtgttccagggcaatcaggactcattcacccctgtggtgaactctctggacccccccctgctgacccggtacctgagaatccacccacagtcttgggtgcaccagatcgccctgaggatggaagtgctgggctgcgaggctcaggatctgtac

SEQ ID NO: 11 - protein, 1438 aa, synthetic construct
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 12 - protein, 1457 aa, synthetic construct
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 13 - DNA, 130 bp, Adeno-associated dependoparvovirus A
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 14 - DNA, 252 bp, synthetic construct
  source 1..252: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 15 - DNA, 60 bp, synthetic construct
  source 1..60: mol_type = other DNA
cgcgaataaaagatctttattttcattagatctgtgtgttggttttttgtgtgatgcagc

SEQ ID NO: 16 - DNA, 141 bp, Adeno-associated dependoparvovirus A
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 17 - DNA, 57 bp, synthetic construct
  source 1..57: mol_type = other DNA
atgcaaatagagctctccacctgcttctttctgtgccttttgcgattctgctttagt

SEQ ID NO: 18 - DNA, 57 bp, synthetic construct
  source 1..57: mol_type = other DNA
atgcagatcgagctgagcacctgcttcttcctgtgcctgctgaggttctgcttcagc

SEQ ID NO: 19 - DNA, 57 bp, synthetic construct
  source 1..57: mol_type = other DNA
atgcagatcgagctgagcacctgtttttttctgtgcctgctgaggttctgcttctcc

SEQ ID NO: 20 - DNA, 57 bp, synthetic construct
  source 1..57: mol_type = other DNA
atgcagatcgagctgagcacctgtttcttcctgtgcctgctgaggttctgtttcagc

SEQ ID NO: 21 - DNA, 57 bp, synthetic construct
  source 1..57: mol_type = other DNA
atgcagatcgagctgtccacctgcttcttcctgtgcctgctgcgcttctgtttctct

SEQ ID NO: 22 - protein, 19 aa, synthetic construct
MQIELSTCFFLCLLRFCFS